(19) United States
(12) Patent Application Publication
(10) Pub. No.: US 2005/0032212 A1
(43) Pub. Date: Feb. 10, 2005

(54) Premeabilisation of cells

(76) Inventor: David Rickwood, Essex (GB)

Correspondence Address: BURNS DOANE SWECKER & MATHIS L L P, POST OFFICE BOX 1404, ALEXANDRIA, VA, 22313-1404, US

(21) Appl. No.: 10/487,086

(22) PCT Filed: Aug. 21, 2002
(86) PCT No.: PCT/GB02/03874

(30) Foreign Application Priority Data
Aug. 21, 2001 (GB) 0120311.6

(51) Int. Cl. C12N 15/00

(52) U.S. Cl. 435/440

(57) ABSTRACT

Provided is a method for permeabilising a viable cell having a cell wall, comprising: (a) pressurising a fluid or gel in contact with a surface of the cell; and (b) depressurising the fluid or gel; to form at least one hole in a surface of the cell.

The present invention relates to a method for permeabilising a cell having a cell wall, and also to a method for introducing a substance into such a cell.

Numerous methods in modern molecular biology and biochemistry require the introduction of various substances into living cells. The introduction of foreign DNA into cells, often resulting in a heritable change in genotype, is termed transfection or transformation. This technique has recently proved to be one of the most important techniques in molecular biology, particularly in relation to genetic engineering and protein engineering. The technique has allowed foreign DNA to be expressed in cells. This is of scientific interest in studying gene transcription and has a wide range of commercial applications involving expressing commercially useful gene products in convenient types of cell.

More recently there has been interest in introducing both proteins and drugs into living cells without damaging the cells. A significant problem to be overcome when developing such techniques is the general imperviousness of the cell membrane. The cell membrane is normally impervious to even small molecules, unless they are have a lipophilic character.

The problem of the imperviousness of the cell membrane is compounded in cells which have a cell wall. The cell wall generally further restricts the movement of substances into the cell, by providing an additional barrier to entry. Prokaryotic cells have a cell envelope which may be defined as a cell membrane and a cell wall, plus an outer membrane if one is present. Gram negative bacteria have a peptidoglycan cell wall composed of protein and polysaccharide, which resides in the periplasmic space between the inner and outer bacterial membranes. The additional outer membrane of Gram negative bacteria further reduces the permeability of the cell envelope. Gram positive bacteria have only a single membrane (analogous to the inner membrane of Gram negative bacteria) but generally have a thicker cell wall. Amongst eukaryotic cells, plant and fungal cells have a cellulose cell wall composed of cellulose microfibrils interwoven with hemicellulose and pectin. The additional strength and reduced permeability provided by the cell wall means that a number of transfection methods which are adequate for animal cells (which do not have a cell wall) are not suitable for cells having a cell wall, such as bacterial, fungal and plant cells.

A number of methods have been devised for permeabilising cells and thereby permitting the introduction of foreign DNA or other substances. Early methods involved binding DNA to particles such as diethylaminoethyl (DEAE) cellulose or hydroxyapatite and adding pre-treated cells which are capable of taking up particles containing DNA. Treatment with calcium chloride, sometimes in combination with low temperature and subsequent heat shock, has commonly been used for the transformation of E. coli. Calcium phosphate co-precipitation provides a general method for the introduction of DNA into mammalian cells. More recently methods have been developed which make use of liposomes loaded with DNA that can be fused with cells. A further technique, termed electroporation, involves subjecting cells to an electric shock which causes the formation of holes in the cells. In Biotechniques, Vol. 17 No. 6 1994, page 118-1125, Clarke et al. disclose a method for introducing dyes, proteins and plasmid DNA into cells using an impact-mediated procedure.

A major problem with the above methods when applied to cells having cell walls is that the uptake of the foreign substance is very inefficient or even virtually undetectable. One way in which this problem has been approached is to remove the cell wall. Prokaryotic and eukaryotic cells with their cell walls removed are typically known as protoplasts.

Protoplasts are generally much more amenable to transformation than cells having cell walls. For instance, Gram-positive bacteria such as Bacillus subtilis can be made more susceptible to plasmid DNA transformation by removing the cell wall (Chang & Cohen, Mol. Gen. Genetics 168, 111-115, 1979). Plant cell protoplasts may be produced by treating suspension cultures, callus tissue or intact tissues with cellulase and pectinase. Transformation of yeast with plasmid DNA was first achieved by using spheroplasts (wall-less yeast cells) from Saccharomyces cervisiae (Hinnen et al. Proc. Natl. Acad. Sci. USA 75, 1929-33, 1978).

However, one disadvantage of using protoplasts is that the cell wall has to be regenerated following the introduction of the substance into the cell. The regeneration medium, in particular for Gram-positive bacteria such as Bacillus subtilis, may be nutritionally complex. Yeast spheroblast cell walls need to be regenerated in a solid agar matrix, making subsequent retrieval of cells difficult. Overall, the process of regenerating cell walls is slow and inconvenient.

Even where protoplasts are used for introducing substances into cells using the methods described above, the efficiency of transfection is often low. In addition, a large proportion of the cells are killed by the above treatments. Even short-term damage to the cell membrane to render it more permeable tends to result in cell-death. This is a particular problem associated with electroporation. Furthermore, in the method of Clarke et al, only a limited number of cells can be transfected in a single treatment.

WO^{o}01/05994 provides a transfection method involving a low incidence of cell death. The method of this document is principally directed to introducing substances into cells by forming holes in the cell membrane using low pressures, generally employing a sparging technique. The document is especially concerned with the transfection of mammalian cells. In particular, the method of WO 01/05994 is preferably applied either to animal cells or to protoplasts in which the cell wall must be removed before transfection. Due to the impaired permeability associated with the cell wall or cell envelope, methods described in WO 01/05994 are not suited to introducing a substance into a cell comprising a cell envelope or cell wall.

There is therefore a need for an improved method of permeabilising a cell having a cell wall. Furthermore, there is a need for an improved method of introducing a substance into a cell having a cell wall.

The present invention aims to overcome the above drawbacks and to provide an efficient method of permeabilising a cell having a cell wall, and thereby permitting entry of a substance such as a nucleic acid into the cell. Accordingly, the present invention provides a method for permeabilising a viable cell having a cell wall, comprising pressurising a fluid or gel in contact with a surface of the cell and then depressurising the fluid or gel thereby forming at least one hole in a surface of the cell.

Without being bound by theory, it is believed that the change in pressure in the fluid or gel causes a warping in the cell membrane, thereby forming a transient hole in the cell membrane. If bubbles are formed due to depressurisation, transient holes may be formed by them and transfection may be achieved. Again without being bound by theory, it is thought that the interaction of the bubbles forming in the proximity of the cell membrane, with the membrane itself, may contribute to the formation of transient holes in the membrane. Therefore, in some embodiments of the present invention, it is preferred that depressurising the fluid or gel generates bubbles of gas which are capable of forming at least one hole in a surface of the cell.

In a further aspect, the present invention provides a method for introducing a substance into a cell having a cell wall, comprising a method for permeabilising a viable cell by a method as defined above, and wherein the at least one hole facilitates entry of the substance into the cell.

The methods of the present invention advantageously allow the formation of transient holes in the cell membrane of the cell, thereby increasing the permeability of the cell to a number of substances. The cell membrane is the plasma membrane which surrounds the cytoplasm, and in the case of Gram negative bacteria refers to the inner membrane lying below the cell wall. The holes formed do not significantly reduce the viability of a significant fraction of the cells, and therefore the incidence of cell death is typically much lower than that associated with a number of prior art methods such as electroporation. The method permits the permeabilisation of cells having cell walls, without the need to completely remove the cell wall as with protoplast-based methods. Furthermore, the cell wall does not need to be regenerated following the procedure as with protoplast-based methods.

This permeabilisation is surprisingly achieved according to the present invention by a pressurisation/depressurisation process. Thus the present invention provides a fast and efficient method of permeabilising a cell having a cell wall. In preferred embodiments of the present invention, bubbles are formed which are thought to contribute to the forming of a hole or pore in the cell membrane of the cell having a cell wall. Without being bound by theory, it is thought that in these embodiments the dimensions of the bubbles, and their composition (in terms of the composition of the fluid or gel and the gas of the bubbles), are sufficient to enable the bubbles to form transient holes in the cell membrane. In all of the embodiments of the present invention, the hole in the cell membrane may comprise a decrease in the thickness of the membrane at a particular point on the surface of the cell, or may comprise the complete removal of the cell membrane from a part of the cell surface. The size of the hole is not particularly limited provided that it increases the permeability of the cell. Preferably the holes should also not be so large such that they deleteriously affect cell function. The hole preferably facilitates the introduction of a foreign substance into the cell, by reducing the barrier to entry provided by the cell membrane.

Typically, the method for permeabilising a cell of the present invention increases the permeability of the cell to a sufficient degree that a foreign substance such as a nucleic acid may be introduced into the cell without a further treatment to increase the permeability of the cell. Alternatively, in certain embodiments the method of the present invention may be combined with one of the prior art methods, such as electroporation or calcium chloride treatment, in order to further increase the efficiency of the method.

The invention will now be further described, by way of example only, with reference to the following Figures, in which:

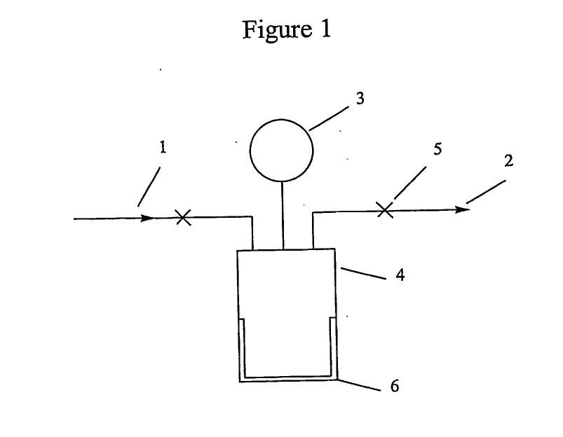

FIG. 1 shows a schematic of the apparatus according to one embodiment of the present invention, wherein 1 is an inlet, 2 is an outlet, 3 is a pressure gauge, 4 is a pressure chamber, 5 is a needle valve and 6 is a coating in the internal surface of the pressure chamber defining a compartment for holding the gel or fluid;

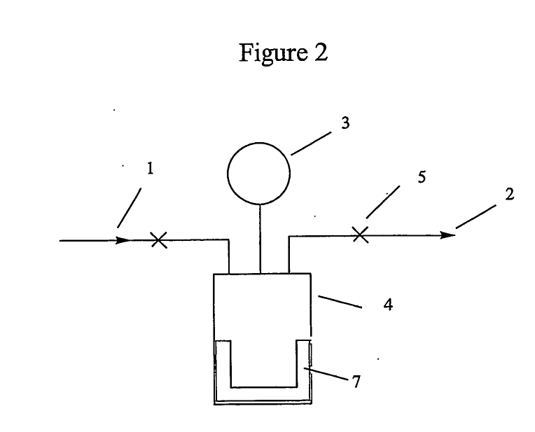

FIG. 2 shows a schematic of the apparatus according to an alternative embodiment of the present invention, wherein 1 is an inlet, 2 is an outlet, 3 is a pressure gauge, 4 is a pressure chamber, 5 is a needle valve and 7 is a receptacle positioned adjacent to an internal surface of the pressure chamber to form a compartment for holding the gel or fluid;

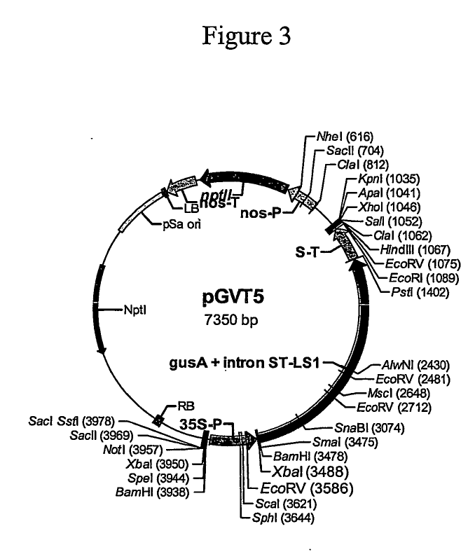

FIG. 3 shows the pGVT5 gene construct;

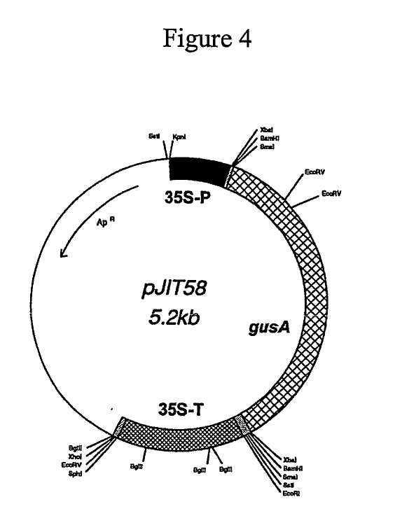

FIG. 4 shows thepJIT58 gene construct;

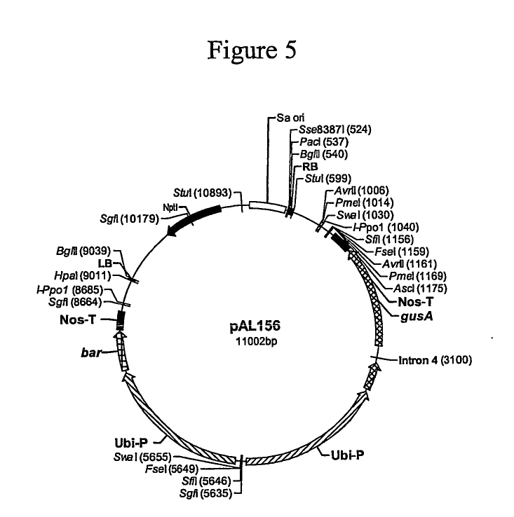

FIG. 5 shows the pAL156 gene construct;

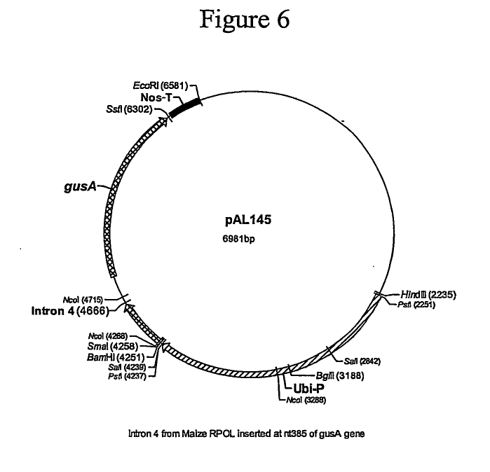

FIG. 6 shows the pAL145 gene construct;

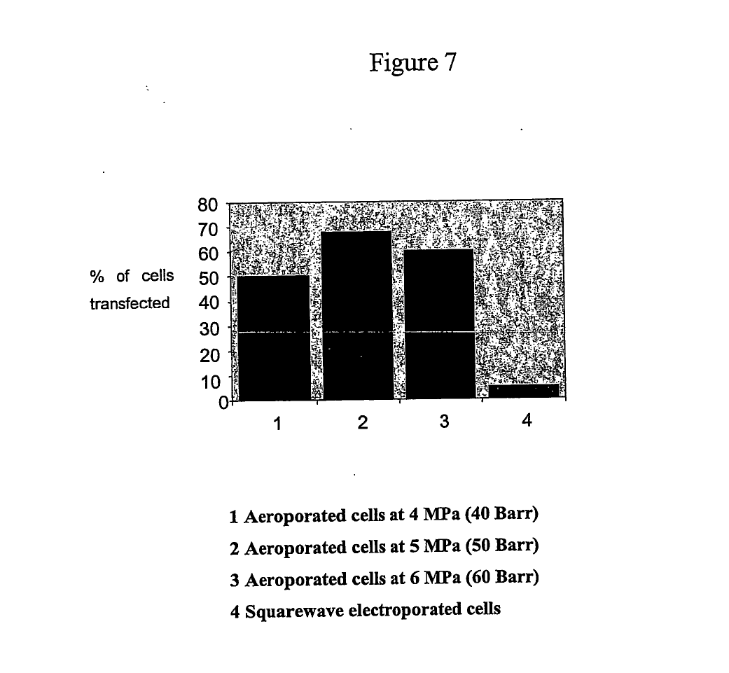

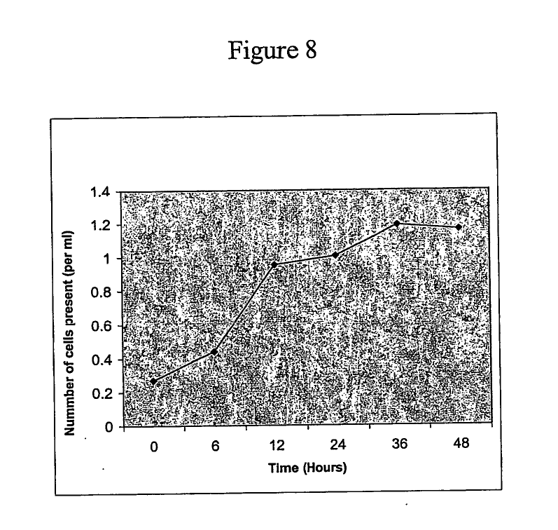

FIG. 7 shows the estimated percentage viability of transfected S. cerevisiae cells; FIG. 8 shows the growth rate of S. cerevisiae cells after aeroporation at 5 MPa (50 Barr);

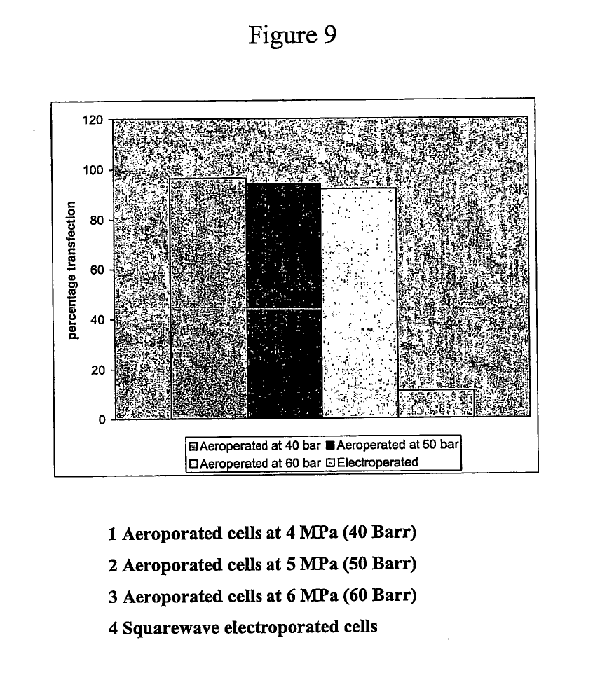

FIG. 9 shows the percentage of cell transfection in yeast cells;

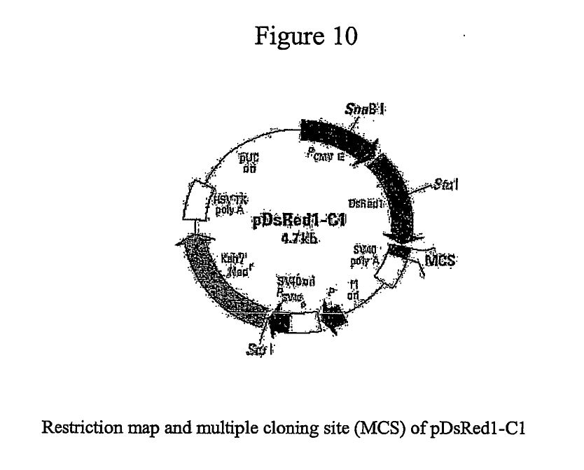

FIG. 10 shows a restriction map and multiple cloning site (MCS) in a red fluorescent protein (RFP) vector, pDsRed1-C1; and

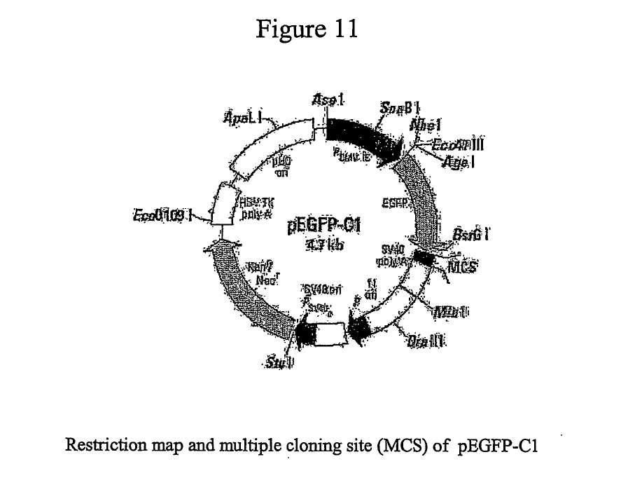

FIG. 11 shows a restriction map and multiple cloning site (MCS) in a green fluorescent protein (GFP) vector, pEGFP-C1.

The more preferred embodiments of the present invention involve the formation of bubbles in the fluid or gel medium. These embodiments and others will now be discussed in more detail.

In the present methods, it has surprisingly been found that cells having a cell wall, which are often more resistant to hole formation than most cells, may be permeabilised by a pressurisation/depressurisation process. As alluded to above, it is thought that depressurisation causes the formation of bubbles within the structure of the cell wall or between the cell membrane and the cell wall. Alternatively, it is thought that bubbles may also form in the interior of the cell, within the circumference bounded by the cell membrane. Bubble formation at such sites may rupture the cell membrane at localised points on the cell surface. The cell wall of the cell is thought to protect the cell membrane against permeabilisation by bubbles forming or bursting outside of the cell wall.

It is believed that other methods where air is introduced into a fluid or gel in order to attempt to permeabilise cells (such as sparging) are ineffective at permeabilising cells having a cell wall, because they do not affect the area between the cell membrane and the cell wall, e.g. by resulting in sufficient warping of the cell membrane due to pressure changes, or bubble formation between the cell membrane and the cell wall.

It is thought that the gas bubbles formed by the depressurisation step of the present method may have sufficient surface energy (or surface tension) that on interacting with the cells (such as contacting the cell membrane and in particular, bursting when in contact with or in close proximity to the cell membrane) a hole is formed in the cell membrane. It is believed to be important that the gas bubbles have a sufficiently small radius that their surface energy is great enough to perforate the cell membrane.

Even though holes are formed in a cell surface according to the method of the present invention, any decrease in cell viability or function is typically less than that observed with the prior art methods. The holes formed in the cells are transient, remaining open for a sufficient time to allow the influx of macromolecules such as DNA and/or RNA into the cell but re-sealing before the viability of the cell is compromised. For instance, using the method of the present invention, cell-death is generally less than 25% and often less than 5%.

In the case of the electroporation procedure, cell-death can be as high as 90%. Even the cells which survive the immediate effects of the procedure may die over the following 24 hours. Typically electroporation results in the immediate death of 50% of the cells by necrosis, followed by the death of most of the remaining 50% of the cells by apoptosis by 24 hours after the procedure. When following the present method, there is typically a low incidence of cell death due to necrosis and/or apoptosis.

Bubbles of gas may be generated in the fluid or gel by a depressurisation process. Depressurisation typically involves reducing the pressure to which the fluid or gel is exposed, such that the solubility of the dissolved gas is reduced, which may cause the formation of bubbles in the liquid. Without being bound by theory, it is believed that the cells in the fluid or gel may act as nuclei for the formation of the bubbles of gas, such that the bubbles form and burst between the cell membrane and the cell wall. Thus the invention advantageously allows the formation, in close proximity with the cell membrane, of bubbles of a suitable surface energy for permeabilising the cell, increasing the efficiency of transfection. Alternatively, as has been mentioned above, the method may cause a perturbation of the cell membrane and/or cell wall due to the pressure change applied, e.g. a warping or distorting of the membrane. Such perturbation may result in the formation of a weak spot in the cell membrane, which may in turn cause a transient rupturing of the membrane. This rupturing may take the form of a transient hole, rip or tear in the membrane, which allows the transfection molecule of choice (e.g. a nucleic acid molecule) to enter the cell.

In the context of the present invention, it is desirable that any dimensions of any bubbles formed during the depressurisation step are controlled such that the bubbles are capable of forming transient holes in the cell (in particular when interacting with a cell surface). The formation of holes in the cell surface using depressurisation and especially using bubbles is termed ‘aeroporation’. Preferably, the dimensions of any bubbles are comparable to the dimensions of the cell. For example, a preferred bubble radius ranges from approximately one third times the radius of the cell to five times the radius of the cell.

According to the present method, the pressurisation step causes an increase in the amount of a gas dissolved in the fluid or gel. The rate of generation of the bubbles of gas, the size of the bubbles and the surface energy of the bubbles may be controlled by varying the rate and extent of the decrease of the pressure in the depressurisation step.

The method typically involves pressurising the fluid or gel and holding the fluid or gel at a starting pressure for a period of time, and then reducing the pressure, preferably to form bubbles. The reduction in pressure is generally 0.5 MPa (5 Barr) or more, and typically within the range of 0.5-11 MPa (5-110 Barr). Preferably it is in the range 1-11 MPa (10-110 Barr), more preferably 2-11 MPa (20-110 Barr), more preferably still 5-11 MPa (50-110 Barr). In some embodiments the pressure reduction may be from 2-8 MPa (20-80 Barr), more preferably 3-8 MPa (30-80 Barr), more preferably still 4-8 MPa (40-80 Barr), and most preferably 6-8 MPa (60-80 Barr). The larger the decrease in pressure in the depressurisation step, the greater the efficiency of hole formation and thus the efficiency of transfection. However, increasing the pressure drop in the depressurisation step may also increase the frequency of damage to the cells leading to cell death. The decrease in pressure may be optimised according to the cell type and the gas which is used, in order to ensure that holes are formed in the cell membrane such that a substance may be introduced, whilst minimising the decrease in cell viability. It is thought that the surface energy of the gas bubbles that may be formed can play a role in the formation of holes in the cell membrane. It is believed that most types of cell having a cell wall may be permeabilised by performing the present invention using a pressure drop within one of the above preferred ranges. The use of a more preferred pressure range will generally tend to increase the proportion of cells which both survive and are transfected.

The starting pressure may be selected to facilitate initial dissolution of gas in the fluid or gel if desired. The starting pressure is generally 0.6 MPa (6 Barr) or more, and typically within the range of 0.6-11.1 MPa (6-111 Barr). Preferably it is in the range 1.1-11.1 MPa (11-111 Barr), more preferably 2.1-11.1 MPa (21-111 Barr), more preferably still 5.1-11.1 MPa (51-111 Barr). In some embodiments the pressure reduction may be from 2.1-8.1 MPa (21-81 Barr), more preferably 3.1-8.1 MPa (31-81 Barr), more preferably still 4.1-8.1 MPa (41-81 Barr), and most preferably 6.1-8.1 MPa (61-81 Barr).

The starting pressure and the pressure decrease to be used may be suitably varied according to (amongst other things) the type of cells to be permeabilised. In one embodiment, where the cells are rice cells, a relatively low starting pressure of 2.1-3.1 MPa (21-31 Barr) is used before depressurising to atmospheric pressure. In another embodiment, where the cells are maize cells, a higher starting pressure of 6.1-7.1 MPa (61-71 Barr) is used.

The length of time the gas is held at the starting pressure is not especially limited, provided that transfection is not adversely affected. Typically, the gas is held at the starting pressure for 1 minute or more, more preferably for 10 mins or more. Generally the pressure is held for less than 30 mins. In some embodiments, the pressure may be held for from 5-20 mins, more preferably from 10-20 mins, and more preferably still for 10-15 mins. It is most preferred that the pressure is held for about 15 mins. This time can be varied, if desired, to alter the quantity of gas initially dissolved in the fluid or gel. The presence of the gas in the fluid or gel can be maintained for as long as necessary, and may be determined according to the conditions employed for permeabilising the cell, such as the gas used, the temperature, the pressure, as well as the type of cell and substance to be introduced into the cell. The efficiency of introduction of the substance into the cell may be particularly sensitive to the length of time the fluid or gel and the cells are exposed to an increased pressure.

The pressure is typically lowered to atmospheric pressure (about 0.1 MPa, 1 Barr). The pressure is preferably lowered rapidly, such as by sudden de-compression, e.g. by exposing the isolated system to the atmosphere. This may be effected by (for example) simply opening a valve or tap connected to the container comprising the fluid or gel. The reduction of pressure preferably takes place over an interval of less than 30 seconds, more preferably less than 10 seconds, and most preferably less than about one second.

The generation of any bubbles of gas that may result from depressurisation may take place continuously for a single period of time or may take place in two or more pulses separated by intervals in which substantially no bubbles are generated. Thus the reduction in pressure may be effected in a single continuous step, or the reduction in pressure may take place in a series of steps of, for example, 0.1-1 MPa (1-10 Barr) separated by intervals in which the pressure is constant.

The cycle of pressurisation and depressurisation may be repeated one or more times. In one embodiment, 2 or 3 pressurisation/depressurisation cycles are used, but preferably only 1 cycle is employed.

In the case where bubbles are generated in pulses, such pulses may typically be from 1-10 s in length. For example, pulses may be from 1-5 s in length, separated by a period of similar length during which no gas generation takes place. Any means may be used for controlling the duration of the pulses. Typically the duration of the pulses may be controlled by a programmable means. Such a means may, for example, include a programmable timer used to control the activity of the means for varying the pressure above the fluid or gel.

The gas used in the present method is not necessarily limited to any one gas in particular, provided that the gas is suitable for pressurising and depressurising the fluid or gel. Preferably the gas is capable of forming bubbles which are able to interact with cells to form transient holes in the cell membrane. A suitable gas may be selected from a wide range of gases including an inert gas, a non-inert gas or a mixture of one or more of both types of gas. Preferably, the gas is air, however oxygen, nitrogen, methane and noble gases such as helium, neon and argon can also be used. In addition, CO₂ can also be used, particularly if it is desirable to maintain the pH of the fluid or gel at a specific level. When CO₂ is used it is generally employed as a 5-7% vol. concentration in another gas, such as air. The gas need not be soluble, but if it is desired to form bubbles in the fluid or gel, the gas should be at least sparingly soluble in the fluid or gel under the conditions at which the method is carried out.

The present method is preferably carried out at a constant temperature, typically at up to 37° C. It is preferably carried out at room temperature, such as from 5-30° C., preferably from 15-30° C.

The pressurisation and depressurisation steps of the present method are carried out in a fluid or gel. The ions present in the fluid or gel are not particularly limited, provided that they can be tolerated by the cells. Where the cell is permeabilised in order to facilitate entry of a substance such as DNA, and the substance is introduced in the same medium, the fluid or gel must also be suitable for the transfection or other introduction process. A transfection medium having an appropriate osmolarity may be formulated using 10 times concentrated Earle's balanced salt solution (EBSS) (Earle, W. R., 1934, Arch. Exp. Zell. Forsch., Vol. 16, p. 116) containing nutrient factors as a base, and diluting as required.

It is preferred that the substance to be introduced into the cell is contained within the fluid or gel. In this preferred embodiment the substance is introduced into the cell in a step which is substantially simultaneous with the step of depressurisation, and (in some embodiments) formation of bubbles in the fluid or gel. However, it is also possible that the substance can be contacted with the cell after depressurisation when the transient hole has been created in the cell surface, provided that the substance is introduced before the transient hole in the cell surface re-seals.

The fluid or gel employed is preferably a liquid, more preferably an aqueous liquid. The liquid may comprise a buffer or a cell culture medium. Preferably the osmolarity of the medium is greater than 100 mOsM. More preferably the osmolarity is from 300-600 mOsM. Using a liquid having an osmolarity within this range tends to reduce cell lysis during the procedure.

In an alternative embodiment where a gel is used, the gel is preferably an aqueous gel. Suitable gels include cell culture media such as agar gels. In this embodiment the cell is typically cultured on the gel.

The concentration of the substance in the medium is not particularly limited and may be selected according to the quantity of substance which is required to be introduced into the cell. A convenient concentration is 0.2-10×10⁻⁸ M, more preferably 0.75-1.25×10⁻⁸ M.

The depth of the fluid or gel is not especially limited. The depth of the fluid or gel is typically 10 cm or less.

The concentration of the cells in the fluid or gel is not particularly limited. For example, the concentration may be of the order of 1×10⁹ cells/ml for prokaryotic organisms.

The substance to be introduced can be any substance. Preferably the substance is a substance not normally able to cross the cell wall and/or cell membrane. It is thus preferred that the substance to be introduced into the cell is a hydrophilic substance, however the substance may also be hydrophobic. Any biological molecule or any macromolecule can be introduced into the cell. The substance generally has a molecular weight of 100 daltons or more. In a more preferred embodiment, the substance is nucleic acid such as DNA or RNA (e.g. a gene, a plasmid, a chromosome, an oligonucleotide, or a nucleotide sequence) or a fragment thereof, or an expression vector. Additionally, the substance may be a bio-active molecule such as a protein, a polypeptide, a. peptide, an amino acid, a hormone, a polysaccharide, a dye, or a pharmaceutical agent such as drug.

The cells to which the method of the present invention can be applied are not particularly limited, in terms of the type of cell or the size of the sample, provided that the cell has a rigid cell wall and is viable. Preferably the cell is a viable live host cell. This includes prokaryotic cells, where the cell wall is part of the cell envelope, and some eukaryotic cells. Thus suitable cells include cells from plants, fungi (including filamentous and non-filamentous fungi such as yeast) and bacteria, including spore-forming bacteria, Gram positive and Gram negative bacteria. The method does not require the formation of protoplasts, and therefore the cell wall is preferably an untreated cell wherein the cell wall has not already been removed, weakened, thinned or perforated prior to the permeabilisation procedure.

Using the method of the present invention, a population of cells can be transfected. These cells may, for instance, be in the form of a cell suspension or may be adherent cells on a solid surface or gel. The method may also be employed to treat a cell population containing a plurality of cell types.

A population of an individual cell type may be permeabilised according to the present method, or a whole tissue, organ or organism may be treated. In one embodiment, the cells are pollen grains, whereas in another embodiment a whole plant is permeabilised. The tissue, organ or organism to be treated may be submerged within the fluid, or alternatively the fluid may come into contact with only a part of the surface of the tissue, organ or organism. In one embodiment, the fluid is sprayed on to the surface of an organ such as the leaf of a plant.

Suitable tissue types comprising cells that may be transfected or transformed according to the methods of the present invention include meristem, disaggregated leaf cells, leaf discs, pollen, microspores (=immature pollen), cotyledon, callous tissue, somatic embryos, pre-embryonic masses, and all suspension culture tissue (=disaggregated cells comprising cell walls).

Where the cells are plant cells, the cells may be from an angiosperm (including a monocotyledon or dicotyledon) or from another order of plants.

The present invention may be used for transformation of any plant species, including, but not limited to, corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea Americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats, barley, vegetables, ornamentals, and conifers.

Preferably, plants of the present invention are crop plants, for example, cereals and pulses, maize, wheat, potatoes, tapioca, rice, sorghum, millet, cassava, barley, pea, and other root, tuber, or seed crops. Important seed crops are oil-seed rape, sugar beet, maize, sunflower, soybean, and sorghum. Horticultural plants to which the present invention may be applied may include lettuce, endive, and vegetable brassicas including cabbage, broccoli, and cauliflower, and carnations and geraniums. The present invention may be applied to tobacco, cucurbits, carrot, strawberry, sunflower, tomato, pepper, chrysanthemum, poplar, eucalyptus, and pine.

Seed-producing plants that provide agronomically-desirable seeds of interest include inter alia oil-seed plants, cereal seed producing plants and leguminous plants. Agronomically-desirable seeds include grain seeds, such as corn, wheat, barley, rice, sorghum, rye, etc. Oil seed plants include cotton, soybean, safflower, sunflower, oil-seed rape, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The present invention may be used for the transformation of any gram-positive or gram-negative bacterium. Suitable gram-positive species include, but are not limited to actinomycetes such as Streptomyces spp., Lactococcus spp., Lactobacillus spp., Bacillus subtilis and Bifidobacter spp. Suitable gram-negative species include, amongst others Escherichia coli and Helicobacter pylori.

The depressurisation means which is employed in the present invention is not particularly limited. A typical depressurisation means comprises a sealable chamber for holding the fluid or gel in which the pressure may be varied and a means for varying the pressure in the chamber. The means for varying the pressure is typically a compressor (such as a cylinder of compressed gas) connected to the sealed chamber, for increasing the pressure in the chamber and/or compressing gas in the chamber. The size and nature of the sealed chamber is not particularly limited provided it is capable of containing the liquid and withstanding a pressure difference between the inside and outside of the chamber. The means for varying the pressure is not particularly limited provided that it is capable of generating a pressure difference between the inside and outside of the chamber.

The depressurisation means may be controlled by a programmable means. Typically a programmable timer is used to control the activity of the depressurisation means.

The container holding the liquid is not especially limited in shape or in the material from which it is constructed, and may be formed from glass or plastics or another convenient material. The container holding the liquid is preferably sealable such that the pressure may be varied, the container being connected to a means for varying the pressure in the container.

Although the means employed to carry out the methods of the present invention are not especially limited, it is preferred that the apparatus set out below is employed. The apparatus of the present invention is an apparatus for introducing a substance into a cell having a cell wall, using a method as defined above, comprising:
(a) an inlet for introducing a gas;
(b) a pressure chamber into which the inlet feeds, which chamber is of substantially geometrical cross section;
(c) a compartment within the pressure chamber for containing the cell in a fluid or gel;
(c) optionally a pressure gauge for monitoring the pressure in the pressure chamber; and
(d) an outlet for releasing gas from the pressure chamber;
   wherein both the inlet and the outlet comprise a valve for isolating the pressure chamber during pressurisation.

Preferably the inlet and outlet comprise inlet and outlet tubes. The diameters of the inlet and outlet are not especially limited, provided that the gas being introduced is capable of pressurising the fluid or gel via the inlet, and that the pressure can be released via the outlet. Preferably the diameter of the inlet and/or the outlet is from 2-4 mm

In the context of the present invention, the term “geometric” referring to the cross-section of the pressure chamber means that the cross section has a substantially uniform geometrical shape, i.e. it is circular (a cylindrical or spherical pressure chamber), square or rectilinear (a cuboidal or rectangular pressure chamber). Preferably, the geometrical cross section of the pressurisation chamber is substantially cylindrical.

In a preferred embodiment, the compartment for containing the cell in a fluid or gel comprises substantially the entire internal surface of the pressure chamber. In this embodiment, the internal surface of the pressure chamber typically comprises a physiologically acceptable coating or layer, such as PTFE (Teflon®), stainless steel or polypropylene. In an alternative embodiment, the compartment for containing the cell in a fluid or gel may comprise a receptacle positioned adjacent to an internal surface of the pressure chamber. In such embodiments, it is preferred that the receptacle is supported by the internal surface of the pressure chamber. Generally the internal surface of the receptacle comprises a physiologically acceptable coating or layer. In the context of the. present invention, this means that the coating or layer is not substantially deleterious to the viability of the cell. Such coatings and layers are well kiown in the art. Preferably the lower portion of the chamber is removable from the upper portion to allow the filling of the chamber or receptacle with the fluid or gel and the cells. This also facilitates cleaning of the chamber and/or receptacle. The chamber may be assembled or disassembled by a screw mechanism or other appropriate mechanisms known in the art.

Typically the valve in the inlet and/or the outlet comprises a needle valve, although the type of valve is not especially limited, provided that it is sufficient to isolate the pressure chamber and control the pressure within it as desired.

In a further aspect, the present invention provides a permeabilised cell comprising a cell wall, obtainable by a method as defined above, wherein the surface of the cell comprises at least one hole which is capable of facilitating the entry of a substance into the cell.

Preferably the hole in the surface of the cell comprises a hole in the cell membrane. Typically there is little damage to the cell wall itself when the present method is performed. Therefore the cell wall of the cell is preferably substantially intact. In a preferred embodiment, the hole is localised such that the cell membrane is substantially intact over at least 50% of the surface of the cell. More preferably, the cell membrane is substantially intact over at least 70% of the surface area of the cell, and most preferably over at least 90% of the surface area of the cell. Preferably the cell membrane of the cell also comprises a hole which is further capable of facilitating the entry of a substance into the cell.

Because the cell wall is relatively undamaged by the present method, it does not need to be regenerated. If it is desired to use the permeabilised cells to introduce a substance therein, it is preferable to introduce the substance substantially simultaneously with or shortly following their production. Alternatively, the permeabilised cells may be stored, typically at −20° C. or below until required and then thawed and used in subsequent procedures.

The present invention also provides use of a depressurisation means to permeabilise a cell and/or to introduce a substance into a cell, wherein the cell has a cell wall and the depressurisation means is used to reduce the pressure applied to a fluid or gel comprising the cell by a step of 2-11 MPa (20-110 Barr).

Life sciences applications in which the present invention can be particularly useful include the introduction of specific genes into viable cells and/or aggregates thereof for expression and for the analysis of the effect of gene products on the metabolism of cells. Such applications also include the expression of biologically active proteins through the introduction of nucleic acid coding for such DNA products into viable cells inter alia to study their effects on the cells with regard to metabolism; protein production; and cell morphology. These applications also extend to the production of pharmacologically important compounds in cells.

In comparison to known methods, the present method is very efficient. The efficiency of transfection depends upon the length of time during which gas generation is carried out, amongst other things. In some circumstances, an efficiency of 80% or more, 90% or more or even approximately 100% can be achieved.

The invention will now be further described, by way of example only, with reference to the following specific embodiments.

### EXAMPLES

### Example 1

### Aeroporation Method for Yeast (Saccharomyces cerevisiae and Schizosaccharomyces. pombe)

5×10⁵ cells grown in yeast-extract mycological growth medium (Oxoid) were washed twice at 1,200 rpm with phosphate-buffered saline (PBS). The pellet was then resuspended in 1M sorbitol. The resuspended cells were transferred to a FACS tube and 0.5 μg of a β-galactosidase DNA vector (pCMV-SPORT-β-gal, Invitrogen) or 2.5 μg of TMR dextran (molecular weight 70,000) Molecular Probes) was added.

The tube was placed in an aeroporator (Baskerville Ltd) and the pressure adjusted over the range of 4-8 MPa (40-80 Barr). The cells were left in the apparatus for one pressurisation/depressurisation cycle of 10 minutes, depressurising to atmospheric pressure.

The cells were then taken out of the aeroporator and washed once with (PBS). The cells were resuspended in 1 ml of liquid media and analysed after 12 hours either by flow cytometry or by fluorescent microscopy (using Poly-L-lysine slides). In the case of TMR dextran, the analysis was done immediately (in order to minimize photo bleaching) and there was no need to resuspend in media.

Trypan blue staining confirmed that the percentage of cells which were viable was greater than 85%. The efficiency of transfection was calculated by the number of cells fluorescing divided by the total number of cells. This gave transfection efficiencies of 60-70%.

### Example 2

### Aeroporation Method for Tobacco Leaf

Tobacco leaf cells in cell culture were counted and the required concentration (0.2-0.5×10⁵ cells/ml) was made up. The cells were centrifuged for 5 mins at 750 g, then the pellet was resuspended in washing medium (phosphate-buffered saline, PBS) and centrifuged again under the same conditions. The pellet was resuspended and centrifuged again.

The pellet was resuspended in 1 ml of transfection medium (MS medium, Sigma, UK) and 0.5 μg of DNA, 2.5 μg of FITC-BSA or 2.5 μg of TMR dextran was added. An aeroporator was connected to a compressed air cylinder, and the cell suspension placed in a sample tube and put into the chamber of the aeroporator.

The lid of the aeroporator was closed and the pressure raised to between 6-8 MPa (60-80 Barr). The cells were left under pressure for 10 mins. After treating the cells, the pressure was released to atmospheric pressure. This pressurisation cycle was repeated 3 times.

The cells were then transferred into a microcentrifuge tube. The cells were washed once with PBS, plated out in the appropriate medium (MS complete medium) and incubated at 25° C.

The cells were then analysed for viability and DNA expression at 5 days post-transfection. Trypan blue staining was used to measure the number of viable cells and efficiency of transfection was calculated by the number of cells fluorescing divided by the total number of cells. The percentage of cells which were viable was 70-80%. The efficiency of transfection was 55-60%.

### Example 3

### Aeroporation Method for Tobacco Root

Tobacco root tip cells in cell culture were counted and the required concentration (0.2-0.5×10⁵ cells/ml) was made up. The cells were centrifuged for 5 mins at 750 g, then the pellet was resuspended in washing medium (phosphate-buffered saline, PBS) and centrifuged under the same conditions. The pellet was resuspended and centrifuged once again in the same way.

The pellet was resuspended in 1 ml of transfection medium (MS medium, Sigma, UK) and 0.5 μg of DNA, 2.5 μg of FITC-BSA or 2.5 μg of TMR dextran was added. An aeroporator was connected to a compressed air cylinder, and the cell suspension placed in a sample tube and pilt into the chamber of the aeroporator.

The lid of the aeroporator was closed and the pressure raised to between 6-8 MPa (60-80 Barr). The cells were left to be treated for 10 mins. After treating the cells, the pressure was released to atmospheric pressure. This pressurisation cycle was repeated 3 times.

The cells were then transferred into a microcentrifuge tube. The cells were washed once with PBS, plated out in the appropriate medium (MS complete medium) and incubated at 25° C.

The cells were then analysed for viability and DNA expression at 5 days post-transfection. Trypan blue staining was used to measure the number of viable cells and efficiency of transfection was calculated by the number of cells fluorescing divided by the total number of cells. The percentage of cells which were viable was 55-60%. The efficiency of transfection was 45-50%.

### Example 4

### Aeroporation Method for Maize Leaf

Maize leaf cells in cell culture were counted and the required concentration (0.2-0.5×10⁵ cells/ml) was made up. The cells were centrifuged for 5 mins at 750 g, then the pellet was resuspended in washing medium (phosphate-buffered saline, PBS) and centrifuged under the same conditions. The pellet was resuspended and centrifuged once again in the same way.

The pellet was resuspended in 1 ml of transfection medium (MS medium, Sigma, UK) and 0.5 μg of DNA, 2.5 μg of FITC-BSA or 2.5 μg of TMR dextran was added. An aeroporator was connected to a compressed air cylinder, and the cell suspension placed in a sample tube and put into the chamber of the aeroporator.

The lid of the aeroporator was closed and the pressure raised to between 6-8 MPa (60-80 Barr). The cells were left under pressure for 10 mins. After treating the cells, the pressure was released to atmospheric pressure. This pressurisation cycle was repeated 3 times.

The cells were then transferred into a microcentrifuge tube. The cells were washed once with PBS, plated out in the appropriate medium (MS complete medium) and incubated at 25° C.

The cells were then analysed for viability and DNA expression at 5 days post-transfection. Trypan blue staining was used to measure the number of viable cells and efficiency of transfection was calculated by the number of cells fluorescing divided by the total number of cells. The percentage of cells which were viable was 60-70%. The efficiency of transfection was 45-50%.

### Example 5

### Aeroporation Method for Maize Root

Maize root cells in cell culture were counted and the required concentration (0.2-0.5×10⁵ cells/ml) was made up. The cells were centrifuged for 5 mins at 750 g, then the pellet was resuspended in washing medium (phosphate-buffered saline, PBS) and centrifuged under the same conditions. The pellet was resuspended and centrifuged once again in the same way.

The pellet was resuspended in 1 ml of transfection medium (MS medium Sigma, UK) and 0.5 μg of DNA, 2.5 μg of FITIC-BSA or 2.5 μg of TMR dextran was added. An aeroporator was connected to a compressed air cylinder, and the cell suspension placed in a sample tube and put into the chamber of the aeroporator.

The lid of the aeroporator was closed and the pressure raised to between 6-8 MPa (60-80 Barr). The cells were left under pressure for 10 mins. After treating the cells, the pressure was released to atmospheric pressure. This pressurisation cycle was repeated 3 times.

The cells were then transferred into a microcentrifige tube. The cells were washed once with PBS, plated out in the appropriate medium (MS complete medium) and incubated at 25° C.

The cells were then analysed for viability and DNA expression at 5 days post-transfection. Trypan blue staining was used to measure the number of viable cells and efficiency of transfection was calculated by the number of cells fluorescing divided by the total number of cells. The percentage of cells which were viable was 55-60%. The efficiency of transfection was 45-50%.

### Example 6

### Aeroporation Method for Rice Leaf

Rice leaf cells in cell culture were counted and the required concentration (0.2-0.5×10⁵ cells/ml) was made up. The cells were centrifuged for 5 mins at 750 g, then the pellet was resuspended in washing medium (phosphate-buffered salne, PBS) and centrifuged under the same conditions. The pellet was resuspended and centrifuged once again in the same way.

The pellet was resuspended in 1 ml of transfection medium (MS medium, Sigma, UK) and 0.5 μg of DNA, 2.5 μg of FITC-BSA or 2.5 μg of TMR dextran was added. An aeroporator was connected to a compressed air cylinder, and the cell suspension placed in a sample tube and put into the chamber of the aeroporator.

The lid of the aeroporator was closed and the pressure raised to between 4-8 MPa (40-80 Barr). The cells were left under pressure for 10 mins. After treating the cells, the pressure was released to atmospheric pressure. This pressurisation cycle was repeated 3 times.

The cells were then transferred into a microcentrifuge tube. The cells were washed once with PBS, plated out in the appropriate medium (MS complete medium) and incubated at 25° C.

The cells were then analysed for viability and DNA expression at 5 days post-transfection. Trypan blue staining was used to measure the number of viable cells and efficiency of transfection was calculated by the number of cells fluorescing divided by the total number of cells. The percentage of cells which were viable was 65-70%. The efficiency of transfection was 55-60%.

### Example 7

### Aeroporation Method for Wheat Leaf

Wheat leaf cells in cell culture were counted and the required concentration (0.2-0.5×10⁵ cells/ml) was made up. The cells were centrifuged for 5 mins at 750 g, then the pellet was resuspended in washing medium (phosphate-buffered saline, PBS) and centrifuged under the same conditions. The pellet was resuspended and centrifuged once again in the same way.

The pellet was resuspended in 1 ml of transfection medium (MS medium, Sigma, UK) and

b 0.5 μg of DNA, 2.5 μg of FITC-BSA or 2.5 μg of TMR dextran was added. An aeroporator was connected to a compressed air cylinder, and the cell suspension placed in a sample tube and placed in the chamber of the aeroporator.

The lid of the aeroporator was closed and the pressure raised to between 6-8 MPa (60-80 Barr). The cells were left under pressure for 10 mins. After pressurising the cells, the pressure was released to atmospheric pressure. This pressurisation cycle was repeated 3 times.

The cells were then transferred into a microcentrifuge tube. The cells were washed once with PBS, plated out in the appropriate medium (MS complete medium) and incubated at 25° C.

The cells were then analysed for viability and DNA expression at 5 days post-transfection. Trypan blue staining was used to measure the number of viable cells and efficiency of transfection was calculated by the number of cells fluorescing divided by the total number of cells. The percentage of cells which were viable was 60-70%. The efficiency of transfection was 20-25%.

The results from examples 2 to 7 are summarised in Table 1 below:

TABLE 1
Effects of aeroporation on cell viability and transfection
efficiency of different plant tissues.

| | Percentage of | Percentage of |
|---|---|---|
| Plant type | viability (%) | transfection (%) |
| Tobacco (leaf) | 70-80 | 55-60 |
| Tobacco (root) | 55-60 | 45-50 |
| Maize (leaf) | 60-70 | 45-50 |
| Maize (root) | 55-60 | 45-50 |
| Rice (leaf) | 65-70 | 55-60 |
| Wheat (leaf) | 60-70 | 20-25 |

In the case of tobacco and maize plants, high pressures were used (6-8 MPa, 60-80 Barr).
In the case of rice plants, lower pressures were used (4-8 MPa, 40-80 Barr).

A similar method to that described in examples 2 to 7 may be applied to other plant species, such as soya and cotton.

### Example 8

### A Comparison between Saccharomyces cerevisiae and Fusarium graminearum Transfection Using High Pressure Aeroporation

The cells selected for this example were yeast Saccharomyces cerevisiae and the filamentous fungus, Fusarium graminearum, which is the myco-protein fungus used to make the food product called Quorn® (Trinci, 1994). This particular filamentous fungus has proven to be difficult to transfect by known methods.

Transfection efficiency is limited by the cell wall, an obstacle that has to be overcome to allow entry of molecules of different sizes and shapes freely into the cell interior. The composition and thickness of the cell wall are important factors that must be considered in determining transfection efficiencies. The cell wall in the yeast S. cerevisiae is in the region of 25% dry cell weight This extracellular mass contributes little to the supportive structure but is necessary for cell protection and control of nutrition, and comprises mostly polysaccharides and glycoproteins with a high proportion of carbohydrates. All of these components have been found in the walls of F. graminearum but the percentage of each making up the wall has not been fully analysed. In most filamentous fungi a polymer of n-acetyl glucosamine called chitin is the major component of the wall. It is also known that the filamentous walls of fungi are generally thicker than the cell walls of yeast. (Wainwright, 1992).

High-Pressure Aeroporation of S. cerevisiae

TABLE 2
Percentages of cell viability and transfection of
S. cerevisiae cells aeroporated at 4, 5 and 6 MPa
(40, 50 and 60 Barr) with and without pEGFP-C1

| | Cell | | | Transfection | | |
|---|---|---|---|---|---|---|
| | Viability | | | (%) at | | |
| | (%) at | | | various | | |
| | various | | | pressures | | |
| | pressures (Barr, | | | (Barr, | | |
| | 0.1 MPa) | | | 0.1 MPa) | | |
| | 40 | 50 | 60 | 40 | 50 | 60 |
| S. cerevisiae | 100 | 98 | 96 | — | | |
| without GFP | | | | | | |
| S. cerevisiae | 100 | 98 | 91 | 57 | 76 | 65 |
| With GFP | | | | | | |

As can be seen from Table 2 transfection was most efficient at 5 MPa (50 Barr) in which 76% of the cells had been transfected, compared to cells aeroporated at 4 MPa and 6 MPa (40 and 60 Barr). This result suggests that high-pressure aeroporation at 5 MPa (50 Barr) permits efficient hole formation in the cell wall which in their turn allows the pEGFP-C1 to enter the cell.

The highest viability percentage was achieved at 4 MPa (40 Barr) in which 100% of the cells survived aeroporation. The lowest percentage was at 6 MPa (60 Barr) in which 91% of the cells survived. These high viability percentages indicate that the process does not seem to be killing the cells or inhibiting their growth cycle. The viability percentage of cells aeroporated at 5 and 6 MPa (50 and 60 Barr) was high, and in the range of 91%-98%.

High-Pressure Aeroporation of F. graminearum

Transfection was most efficient at 6 MPa (60 Barr). Good fluorescence was observed when the mycelium was subjected to 6 MPa (60 Barr).

In carrying out aeroporation, a thin 1 cm² fragment of the mycelium was cut out and aeroporated at 6 MPa (60 Barr). Fluorescence occurred throughout the complete length of this fragment.

High-Pressure Aeroporation of F. graminearum Using 1 and 2 Cycles

The application of more than one cycle at the same pressure allowed an increase in transfection to occur. This was also seen in similar experiments carried out in which the F. graminearum aeroporated at 7 MPa (70 Barr) and processed with 2 cycles showed better fluorescence compared to lower pressures also using 2 cycles.

### Example 9

### Aeroporation Procedure for Plant and Fungal Suspension Cells

Macromolecules Used for Cell Transfection

The macromolecules used for transformation were mainly fluorescent probes since they can be detected using fluorescent microscopy and flow cytometry.

Macromolecules Used During This Project were:
TMR-Dextran (tetramethyl rhodomine dextran) (mol. wt. 70,000 Da)
GFP DNA Vector (green fluorescent protein DNA) (4.76 kb) (PEGFP)
β-gal DNA (8.2 kb)
   TMR-Dextran

TMR-dextran is a polysaccharide covalently linked to TMR, a fluorescent-labelled reagent. Molecular weights of 10,000, 40,000, and 70,000 and diameter of 5.4 nm dextrans were used. TMR-dextran is used widely as a molecular marker (Hougland., 1996). Excitation wavelength was at 546 nm when using a flow cytometer.

Analysis of Cells

Cells were analysed using spectroscopy, gel electrophoresis, flow cytometry, light and fluorescent microscopy.

Method and Conditions of Cell Growth

Growth Conditions of Yeast Cells

Both S. cerevisiae and S. pombe were grown on pre-prepared Malt extract agar (Oxoid) agar plates, and grown at 25° C. in a cooled incubator for 48 hours. Colonies were then picked off using sterile tooth picks and used to inoculate yeast malt extract liquid media (YME—10 g of glucose, 5 g of peptone, 3 g of yeast extract and 3 g of malt extract and made up to 1 litre with double distilled water, then autoclaved). Inoculated cultures were grown to exponential phase in a cooled orbital shaker at 25° C. and then transfected.

Growth Conditions of Filamentous Fungi (Fusarium graminarium)

Fusarium graminarium was grown on potato dextrose agar (Oxoid) by subculturing 1 cm of the organism on solid medium for 7 days at 25° C. After 7 days a malt extract or Czapex dox liquid media was inoculated with a 1 cm piece of Fusarium and grown at 25° C. for 5 days. After 5 days the Fusarium was strained through a sterilised filter funnel with Whatman number 1 filter paper. The mycelium were cut into approximately 2 cm pieces, washed and transfected.

Transfection and Washing Solution

1 M Sorbitol was used as the transfection medium and 1×PBS was used as washing medium.

Method of Cell Transfection Using High Pressure Aeroporation

Cells were counted (approximately 0.5-1×10⁶ cells/ml)
Wash cells in 1 ml of sterile dd. H₂O by centrifuging at 1300 rpm for 5 mins (twice)
Wash in ice cold 1× Phosphate buffered saline (PBS)
Resuspend cells in cold 1 M sorbitol and transfer into a FACS tube
Add 0.5 μl of macromolecules into the solution
Put tube into the aeroporator and close the chamber
Close the air out let and adjust pressure as required
Open the air inlet and allow pressurisation to take place for 15 mins
Depressurise the chamber by closing the inlet and opening the outlet
Open chamber and remove FACS tube
Spin cells at 1300 rpm and then resuspend in media and allow cells to grow to exponential phase (if Dextrans are being use analysis should be done immediately after aeroporation.
Prepare cell for analysis.
   Preparation of Cells for Analysis After Transfection
   GFP Transfected Cells Analysis by Fluorescent Microscopy
Count cells
Wash with 1×PBS
Resuspend in 2 μl of 1×PBS
And add solution containing cells to Poly-L-lysine multi welled slides.
Leave slide for 20 mins
Take liquid off by aspiration.
Add 2 μl of 1×PBS to slide and leave for 5 mins
Remove PBS
Add a drop of DABCO to the slide and carefully place a cover slip on the slide.
Analyse by Fluorescent Microscopy
   Analysis of GFP Treated Cells by Flow Cytometry
Wash cells at 1300 rpm with 1×PBS (twice)
Resuspend in 1 M sorbitol and taken to the flow cytometer for analysis.
   Analysis of β-Galactosidase Expression in the Cell

Analysis was carried out by incubating treated cells on a diagnostic slide treated with β-Gal buffer for 24 hours and then viewing under phase contrast.

Analysis of Viability and Percentage Transfection

The percent viability was obtained by growth curves before and after aeroporation and the use of Trypan blue. The percentage transfection was worked out using flow cytometry

Results of Transfection

Transfection of Yeast Cells

The transfection of S. cerevisiae and S. pombe using the aeroporator is simple yet effective.

Air was used at pressures between 34 MPa (30-40 Barr) for the aeroporation experiments of both types of yeast cells. All cells were transfected for 1 cycle lasting 15 mins.

Efficient transfection was achieved best at 5 MPa (50 Barr), the percentage transfection was very high and the percentage viability was also high (Tables 3 and 4).

Transfection of Filamentous Fungi

Transfection of filamentous fungi was done using air at 3-7 MPa (30-70 Barr) (e.g. 6 MPa, 60 Barr) for one 15 minute cycle. Indications are that, these cells will also transfect at higher pressures (7-8 MPa, 70-80 Barr) using more than one cycle.

Comparison of transfection of yeast cell by aeroporation and square wave electroporafion (Table 3) indicates that aeroporation is more efficient for these cells.

TABLE 3
Transfection efficiency and viability with the
use of aeroporation (A) and no aeroporation
(NA) for Saccharomyces cerevisiae

| | % of cell transfection | | | % Viability of cells 24 hours | | |
|---|---|---|---|---|---|---|
| | at various pressure | | | after transfection at various | | |
| | (10 × MPa/Barr) | | | pressures 10 × MPa/Barr) | | |
| | 40 | 50 | 60 | 40 | 50 | 60 |
| Cells + | 58-64 | 65-72 | 58-66 | 97-100 | 96-98 | 94-98 |
| pEGFP (A) | | | | | | |
| Cells only | 0 | 0 | 0 | — | — | — |
| (NA) | | | | | | |
| Cells only | 0 | 0 | 0 | 97-99 | 94-96 | 94-98 |
| (A) | | | | | | |
| Cells + | 52-68 | 60-69 | 54-56 | 98-100 | 94-96 | 94-98 |
| pEGFP (A) | | | | | | |
| Cells + β- | 0 | 0 | 0 | — | — | — |
| gal (NA) | | | | | | |
| Cells + 70K | 55-60 | 70-74 | 69-70 | 97-100 | 94-96 | 98-100 |
| Dex (A) | | | | | | |
| Cells + 70K | 0-0.5 | 0-0.5 | 0-1 | — | — | — |
| (NA) | | | | | | |

TABLE 4
Transfection efficiency and viability with the use of
aeroporation (A) and no aeroporation (NA) for
Schizosaccharomyces pombe

| | | | | Estimated % | | |
|---|---|---|---|---|---|---|
| | | | | viability of cells 24 | | |
| | Estimated % of cell | | | hours after transfection at | | |
| | transfection at various | | | various pressures | | |
| | pressure (10 × MPa/Barr) | | | (10 × MPa/Barr) | | |
| | 40 | 50 | 60 | 40 | 50 | 60 |
| Cells + pEGFP | 50-52 | 66-70 | 58-61 | 96-97 | 93-95 | 90-94 |
| (A) | | | | | | |
| Cells only | 0 | 0 | 0 | — | — | — |
| (NA) | | | | | | |
| Cells only | 0 | 0 | 0 | 95-97 | 91-94 | 90-91 |
| (A) | | | | | | |
| Cells + pEGFP | 50-51 | 56-59 | 54-57 | 94-95 | 90-92- | 90-91 |
| (A) | | | | | | |
| Cells + β- | 0 | 0 | 0 | — | — | — |
| gal (NA) | | | | | | |
| Cells + 70K | 66-68 | 68-70 | 54-56 | 95-97 | 92-93 | 89-94 |
| Dex (A) | | | | | | |

TABLE 5
Electroporated cell viability and efficiency of
yeast cells with pEGFP-C1 vector

| | % viability after electroporation | | | % cells |
|---|---|---|---|---|
| Samples | 12 hrs | 24 hrs | 48 hrs | transfected |
| S. pombe + DNA | 40-50 | 20-30 | Less than 5 | 5.5-16 |
| vector | | | | |
| S. cerevisiae + DNA | 40-50 | 35-37 | Less than 7 | 12.8-20 |
| vector | | | | |

TABLE 6
Time after aeroporation at 4 MPa (40 Barr) when yeast
cells cease incorporating macromolecules.

| Time in seconds | S. cerevisiae | S. pombe |
|---|---|---|
| 20 | Yes | Yes |
| 40 | Yes | Yes |
| 60 | Yes | Yes |
| 90 | Yes | No |
| 120 | No | No |

TABLE 7
Time after aeroporation at 5 MPa (50 Barr) when
yeast cells cease incorporating macromolecules.

| Time in seconds | S. cerevisiae | S. pombe |
|---|---|---|
| 20 | Yes | Yes |
| 40 | Yes | Yes |
| 60 | Yes | Yes |
| 90 | No | No |
| 120 | No | No |

It was determined that transfection of yeast was most efficient at 5 MPa (50 Barr) using the high-pressure aeroporation. Transfection efficiency remains very high even at high pressure (Tables 3 and 4; FIG. 9) without significant loss of viability with the use air.

The percentage viabilty of both S. pombe and S. cerevisiae remained high even after 48 hours using aeroporation indicating that this process does not seem to kill the cells or inhibit their growth cycle (FIG. 8). However, transfection with the square wave that the system seems to disrupt the cell, the percentage yield and viability being lower (FIG. 7; Table 5).

Indications are that transfection of yeast by aeroporation is much more efficient than electroporation. Experiments carried out to explore the time it takes for the holes in the cell wall to re-seal showed that in both species of yeast the holes re-sealed much faster at 5 MPa (50 Barr) than at 4 MPa (40 Barr) in air. Experiments done with aeroporation also showed that Fusarium responded positively to aeroporation at 6-7 MPa (60-70 Barr).

The work shows that aeroporation although a fairly simple method is a very effective and very advantageous method for transfection.

References

Bell H., Kimber W. L., Li M., Wittle I. R, Neuroreport, 9(5), pp.793-798,1998
Fenton M., Bone N., Sinclair A. J., Journal of Immunological Methods, 212(1), pp41-48, 1998.
Mascarenhas L., Stripecke R., Case S. S., Xu D. K., Weinberg K. I., Kohn D. B., Blood, 92(10), pp3537-3545, 1998.

### Example 10

### Aeroporation Method for NT1 and BMS Cell Cultures

Materials and Methods for the Culturing/Maintenance of the NT1 and BMS Cell Cultures

BMS (Black Mexican Sweet) maize (Zea mays L.) cell suspension was obtained from the John Innes Centre (Norwich, UK). BMS cell suspension was cultured as previously described by Green C. E. (1977), ‘Prospects for crop improvement in the field of cell culture’, Hort. Science 12:131-134.

NT1 tobacco (Nicotiana tabacum L.) cell suspension was obtained from the John lines Centre (Norwich, UK). NT1 cell suspensions were cultured as previously described by Fromm M, Callis J, Taylor L P, Walbot V (1987) Methods Enzymol. 153:351-366.

The following gene constructs were used at the University of Essex:
PJIT58 (P. Mullineaux, JIC) for plant cell transformation (see FIG. 4)
PAL145 (D. Lonsdale, JIC) for plant cell transformation (see FIG. 6)
PGVT5 (V. Thole, JIC) for NT1 tobacco CS transformation (see FIG. 3)
PAL156 (D. Lonsdale, JIC) for BMS CS and rice ECS transformation (see FIG. 5)

All the above gene constructs are publicly available and can be obtained from the John Innes Centre (Norwich, UK).

Detail of the gene constructs is provided in maps:
gusA: glucuronidase gene from E. coli
bar: from Phosphinitricin acetyltransferase gene from Streptomyces hygroscopicus
nptII: Neomycin phosphotranspherase gene from E. coli
Intron 4: intron 4 from Zea mays phage type polymerase gene
Intron ST-LS1: intron 2 of ST-LS 1 gene from Solanum tuberosum
35S-P: 35S promoter from Cauliflower Mosaic Virus
Ubi-P: Ubiquitin 1 promoter+exon1+intron 1 from Zea mays
nos-P: nopaline synthase promoter from Agrobacterium
35S-T: polyadenylation sequence from Cauliflower Mosaic Virus
S-T: polyadenylation sequence from Glycine max.
nos-T: nopaline synthase polyadenylation sequence from Agrobacterium

### 10a

### Culturing/Maintenance and Preparation of Rice Embryogenic Cell Suspension Cultures Prior to Aeroporation

Production of Embryogenic Rice Callus

Mature seeds of rice (Oryza sativa L.) variety Nipponbare were used for callus production using modified protocols from Sivamini et. al. 1996, Wang et. al. 1997 and Bec et. al 1998. Dehusked seeds were sterilised with half strength commercial bleach for 15 min and rinsed three times with sterile distilled water. The embryos were aseptically removed under a dissecting microscope and plated onto NBm medium (macro-element N6, micro-elements B5, Fe-EDTA, 30 g 1⁻¹ sucrose, 30 g 1⁻¹ 2,4-D 2 mg 1⁻¹, 300 mg 1⁻¹ hydrolysate, 500 mg 1⁻¹ L-glutamine, 500 mg 1⁻¹ L-proline, 2.5 g 1⁻¹ Phytagel, pH 5.8, filter-sterilized vitamins B5 added after autoclavage) for 3 weeks in the dark at 25° C. Loose embryogenic translucent globules (U), around 1 mm in size, were separated from the original embryo onto the gelling agent. Globules were cultured for an additional 10 days onto fresh NBm medium to produce embryogenic nodular units (ENU, Bec et. al. 1998).

Production of Embryogenic Cell Suspensions (ECS)

Embryogenic nodular units (ENU) were dispersed in 250 ml flask containing 40 ml NBm liquid medium, shaken at 100 rpm at 25° C. in the dark. Every week, old culture medium was removed from each flask and ˜500 μl PCV cells were subcultured into new flask containing 40 ml fresh NBm liquid medium.

Preparation of Rice ECS for Aeroporation

One week old rice ECS were filtered through a 1 mm nylon mesh. Aliquots of filtrate were used for aeroporation (in experiment 30/7/02˜50 μl PCV rice cells in 0.2 , 0.5 or 1 ml NBm liquid medium).

References

Sivamani, E., Shen, P., Opalka, N., Beachy, R. N. and Fauquet, C. M. (1996) Selection of large quantities of embryogenic calli from indica rice seeds for production of fertile plants using the biolistic method. 15: 322-327

Bec, S., Chen, L., Ferriere, N. M., Legave, T., Fauquet, C. and Guideroni, E. 1998. Comparative histology of microprojectile-mediated gene transfer to embryonic calli in japonica rice (Oryza sativa L.): influence of the structural organization of target tissues on genotype transformation ability. Plant Science 138: 177-190. Wang, M. B., Upadhyaya, N. B., Brettell, R. I. S. and Waterhouse, P. M. 1997. Intron-mediated improvement of a selectable marker gene for plant transformation using Agrobacterium tumefaciens. J Genet & Breed 51: 325-334.

### 10b

### Transfection of Suspension BMS and NT1 Plant Cells Using Aeroporation

Transfection refers to a range of techniques used for introducing specific double stranded DNAs into dividing eukaryotic cells in such a way that they can be taken up by the nucleus and expressed.

It was found that suspension cultures of plant cells could be transfected using high pressure aeroporation.

This example describes work carried out to study the transfection of BMS and NT1 cells using high-pressure aeroporation.

Transfection Procedure

BMS and NT1 suspension plant cells cultured in the appropriate media were used for the experiments. Cells were transfected by aeroporation using 1 cycle of pressurisation/depressurisation to 6-7 MPa (60-70 Barr) for 15 minutes as previously described.

Reporter Molecules

For this set of experiments different reporter DNA vectors have been used. These include β-glucuronidase (pAL145, RT18 for BMS cells and PJIT58, PGVT5 for NT1 cells. All plasmids used were provided by the John Innes Centre). Green fluorescent protein vector (GFP) has also been used.

Finally both BMS and NT1 suspension plant cells were transfected with TMR-Dextran (70,000 MW) using the aeroporator.

Culture of Cells

BMS Cells

These were cultured in BMS suspension cell medium Cells were subcultured weekly. 10 ml of culture plus 50 ml of fresh medium were added in a 250 ml flask. The cells were shaken at 150 rpm at 25° C.

NT1 Cells

These were cultured in NT1 suspension cell medium Cells were subcultured as 1:50 and 1:100 dilutions every week. They were shaken at 125 rpm at 25° C., shaded with foil.

Rice Embryogenic Cell Suspension Cultures

They were cultured in NBm medium. The cells were subcultured weekly. They were shaken at 1000 rpm, 25° C. in the dark.

Cell Analysis

Light Microscopy—Bright-Field Microscopy

Bright-field microscopy is the most widely used technique in the field of light microscopy. Normally, living single cells or monolayers of cells are almost invisible in an ordinary light microscope. When supplemented by stains though, bright-field microscopy is a powerful technique.

Light Microscopy—Fluorescence Microscopy

Fluorescence microscopy is based on the property of some substances to absorb light in a certain wavelength range and then to emit it in the form of light For our studies and Olympus IM12 microscope was used. For our fluorescent proteins it was possible to use the normal FITC filter.

Results

Transfection of cultured BMS cells with GFP vector using the aeroporator (B) at 7 MPa (70 Barr) for 15 minutes. Significant fluorescence was observed in test cells. Untreated controls showed no fluorescence.

Transfection of cultured NT1 Cells with GFP Vector

Cells were treated for 15 mins at 6 Pa (60 Barr) and 7 MPa (70 Barr), respectively. Significant fluorescence was observed in test cells. Untreated controls showed no fluorescence.

Transfection of Cultured BMS Suspension Cells with TMR-Dextran (70,000 MW)

The cells were treated in the aeroporator for 15 mins at 7 MPa (70 Barr). Significant fluorescence was observed in test cells. Untreated controls showed no fluorescence.

Transfection of Cultured NT1 Cells with TMR-Dextran (70,000 MW)

The cells were treated in the aeroporator for 15 min (1 cycle) at 7 MPa (70 Barr). Significant fluorescence was observed in test cells. Untreated controls showed no fluorescence.

Stable Transfection of Cultured NT1 Cells with PGVT5 Vector (GUS) as Per Previous Experiments (i.e. 1 Cycle: 15 mins at 6-7 MPa (60-70 Barr))

Pictures taken after culture for about 2 weeks in selective medium, and after a further 2 and 3 weeks of culture in non-selective medium showed significant staining in test cells.

Transfection of Cultured Rice Embryogenic Cultures with TMR-Dextran and GFP

Test rice embryogenic cells showed significant blue colouring. Untreated controls showed no fluorescence.

### Example 11

### Materials and Methods for the Subculturing and Selection of Cells Following Transformation with Aeroporation

Following aeroporation treatment, rice ECS were plated onto a Whatman filter on a petri dish containing the NBm solid medium and cultured for 2 days in the dark at 25° C.

Two days after aeroporation, filters were transferred onto selection medium (NBm solid medium plus either 5 mg/l phosphinotrycin (PPT, selection pAL156) or 100 mg/l geneticin (selection pGVT5) for 2 weeks in the dark at 25° C. L-glutamine was removed from all culture media when PPT was included.

Two weeks after transformation, each callus (grown from an individual ENU was split into 2 to 5 pieces. Pieces of callus were cultured for 3 additional weeks onto fresh NBm-based selection medium The resistant calli grown from individual ENU, after 2+3 weeks selection, were all grouped together.

Five weeks after aeroporation, the resistant calli were transferred to PRm pre-regeneration medium (NBm solid medium without 2,4-D but with 2 mg/l BAP, 1 mg/l NAA, 5 mg/l ABA plus either 5 mg/l PPT (selection pAL156) or 100 mg/l geneticin (selection pGVTS)) for 9 days in the dark at 25° C.

Six weeks after aeroporation, calli showing clear differential growth were then transferred to regeneration medium RNm (NBm medium solid without 2,4-D but with 3 mg/l BAP, 0.5 mg/l NAA plus either 5 mg/1 PPT (selection pAL156) or 100 mg/l geneticin (selection pGVT5)) for 2-3 weeks in the light at 25° C. Only one plant was regenerated from each original ENU to guarantee that each plant represented an independent transformation event.

Eight to nine weeks after aeroporation, plants were developed on MSR6 solid medium (Vain et al. 1998) containing either 5 mg/l PPT (selection pRT18) or 100 mg/l geneticin (selection pGVTS) for 2-3 weeks at 25° C. in the light.

Ten to twelve weeks after aeroporation, transformed plants were transferred to a controlled environment room for growth to maturity and seed setting.

GusA gene activity was monitored in rice calli and plants during the selection process by histochemical GUS staining following the method of Jefferson (1987). Molecular analysis of the transformed plants was performed using PCR and Southern blot analysis.

References

Jefferson R A, Kavanagh T A, Bevan M W (1987) β-glucuronidase as a sensitive and versatile fusion marker in higher plants. EMBO J. 6: 3901-3907

Vain, P., Worland, B., Clarke, M. C., Richard, G., Beavis, M., Liu, H., Kohli, A., Leech, M., Snape, J. W., Christou, P., and Atkinson, H. 1998. Expression of an engineered proteinase inhibitor (Oryzacystatin-IΔd86) for nematode resistance in transgenic rice plants. Theor. and Appl. Genet 96: 266-271.

TABLE 8
MEDIUM NBm liquid

| | NBm liquid - 1 L | NBm solid - 1 L |
|---|---|---|
| Macro N6 | 100 ml | 100 ml |
| Micro B5 | 10 ml | 10 ml |
| FE-EDTA | 10 ml | 10 ml |
| Sucrose | 30 g | 30 g |
| 2,4-D | 2 mg | 2 mg |
| Caseine hydrolysate | 300 mg | 300 mg |
| L-Glutamine | 500 mg | 500 mg |
| L-Proline | 500 mg | 500 mg |
| Phytagel | — | 2.5 g |
| make up to volume | 990 ml | 990 ml |
| PH (with KOH) | 5.8 | 5.8 |
| Add after autoclavage | | |
| vitamin B5 | 10 ml | 10 ml |
| (PH 5.8, sterile) | | |

Stock MACRO N6 (Chu et al 1975)

For 1 l

| | |
|---|---|
| KNO₃ | 28.3 g |
| (NH₄)₂SO₄ | 4.63 g |
| CaCl₂ 2H₂O | 1.66 g |
| MgSO₄ 7H₂O | 1.85 g |
| KH₂PO₄ | 4 g |

Make up to volume 1 l

| | |
|---|---|
| Utilisation: | 100 ml/l of medium |
| Storage: | 4° C., not sterile |

Stock MICRO B5 (Gamborg et al. 1968)

For 500 ml

| | |
|---|---|
| H₃BO₃ | 150 mg |
| MnSO₄.4H₂O | 660 mg |
| ZnSO₄.7H₂O | 100 mg |
| KI | 37.5 mg |
| Na₂MoO₄.2H₂O | 12.5 mg |
| CuSO₄.5H₂O | 1.25 mg (5 ml of 0.25 mg/ml stock) |
| CoCl₂.6H₂O | 1.25 mg (4.5 ml of 0.28 mg/ml stock) |

Make up to volume 500 ml

| | |
|---|---|
| Utilisation: | 10 ml/l of medium |
| Storage: | 4° C., not sterile |

Stock VITAMINS B5 (Gamborg et al. 1968)

For 500 ml

| | |
|---|---|
| Thiamine HCl | 500 mg |
| Pyridoxine HCl | 50 mg |
| Nicotinic acid | 50 mg |
| Myo-inositol | 5 g |

Make up to volume 500 ml

| | |
|---|---|
| Utilisation: | 10 ml/l of medium |
| Storage: | PH 5.8, 4° C., filter sterilized, dark container |

References
Gamborg O L, Miller R A, Ojima K (1968) Requirements of suspension cultures of soybean root cells. Exp Cell Res. 50:151-158
Chu C C, Wang C C, Sun C S Hus C, Yin K C, Chu C Y, Bi F Y, (1975) ‘Establishment of an efficient medium for anther culture of rice through comparative experiments on the nitrogen sources.’ Sci. Sin. (Pekin) 18:659-668.

### Example 12

### Stable Transfection of Plant Cells Using Aeroporation

Suspension cultures of plant cells can be transfected using high-pressure aeroporation. However, many of the cells express the transfected vector itself, which is not integrated into the host genome, and this is known as transient expression.

Generation of cells in which foreign genes are stably incorporated into the host genome requires a method of selection of transfected from non-transfected cells. This is usually carried out by co-transfecting into the cells a gene for constitutive expression of a gene conferring antibiotic resistance on the transfected cells. The antibiotic resistance gene is preferably carried on the same plasmid vector as the foreign gene of interest. One commonly used method of selection is to use the neomycin gene, which confers resistance to G418 sulphate in recipient cells. This report describes work carried out to study the stable transfection of cells using high-pressure aeroporation.

Transfection Procedure

Suspensions of plant cells were derived from chopped tobacco and maize leaves by culture for at least three days in either MS or B5 medium were used for all experiments. Cells were transfected by aeroporation using one cycle of pressurisation/depressurisation to 7 MPa (70 Barr) as previously described.

Reporter Molecules

Four different types of reporter DNA vectors have been used for plant cell transfection studies and these include β-galactosidase (β-gal), glucuronidase (GUS), green fluorescent protein vector (GFP) and red fluorescent protein vector (RFP).

GFP is useful because it can be detected without killing the cells. Cells transformed with the GFP gene exhibit bright fluorescence. GFP is a highly stable protein with a small molecular weight and shows very little photobleaching. This reporter system has been shown to function in a wide variety of biological systems, including plants (Corbett, 1995; Haseloff, 1995; Kaether, 1995; Wang, 1994). On the other hand, the RFP shows no autofluorescence.

The advantages of GFP and RFP is that cells that express the reporter gene can be identified through fluorescence microscopy and this enables the cells to be sorted using flow cytometry. Both vectors also have the neomycin gene making it easy to select for transfected cells in culture. For this reason the first experiments have been carried out using GFP and RFP DNA vectors using a concentration of 2 μg/ml.

Culture of Cells

Immediately after transfection cells were cultured in MS medium but with the addition of geneticin (G418) (Sigma) to select for cells that have been transfected because of the presence of the neomycin resistance gene. Before beginning, a dose-response curve of cell death by the selection antibiotic was performed on the cells to be transfected. It was important to use the correct concentration of selection medium, which would be just enough to kill most of the untransfected cells over a 1-3-day period. During our experiments we used 1000 μg/ml of G418 for full selection. Cells were grown in this selective medium for at least 2-3 weeks, changing the medium as required every 3-4 days and the cells were then transferred to non-selective medium for further growth.

Cell Analysis

Fluorescence-Activated Cell Sorting

This technique can be used to separate cells on the basis of their light-scattering properties and the particular surface molecules, which they express. These molecules can be detected by the use of specific ligands (e.g. antibodies) labelled with a fluorochrome. A stream of microdroplets containing the cells is passed through a laser beam. Light scattering at low angle and at 90° is detected, along with the fluorescence of the fluorochrome excited by the laser. Cells with light scattering and fluorescence parameters falling within predetermined limits are electrostatically deflected for collection. The technique can also be adapted to deflect single cells into the wells of multi-well plates.

Fluorescence Microscopy

Cells were examined using either bright-field microscopy or fluorescence microscopy using an Olympus IMT2 microscope. For both fluorescent proteins it was possible to use the normal FITC filter.

Results

GFP Pictures from Stable Transfection of Cultured Tobacco Leaf Cell Cultures

Stable transfection of cultured tobacco cells with GFP vector using the aeroporator. Pictures were taken after culture for 2 weeks in selective medium and after a further 2 weeks of culture in non-selective medium Untreated controls showed no fluorescence whereas transfected cells showed significant fluorescence.

RFP Pictures from Stable Transfection of Cultured Maize and Tobacco Leaf Cells

Stable transfection of cultured maize, and tobacco cells. The cells were transfected with RFP vector using the aeroporator. Pictures were taken after 2 weeks of culture in selective medium. Untreated controls showed no fluorescence whereas transfected cells showed significant fluorescence.

From our experiments, it is quite clear that stable transfection can be readily effected using the aeroporation technology. Cultures of tobacco leaf cells stably transfected with GFP vector have been growing successfully for about 4 weeks: 2 weeks in selective medium and for a further two weeks in non-selective medium. As in the case of the GFP vector the level of expression of RFP appears to be lower in maize cells compared with tobacco.

References

Corbett, A. H., Koepp, D. M., Sclenstedt, G., Lee, M. S., Hoper, A. K., Silver, P. A. (1995). Rna1p, a Ran/TC4 GTPase activating protein, is required for nuclear import. J. Cell Biol. 130, 1017-1026
Haseloff, J., Amos, B. (1995) GFP in plants. TIG 11,328-329
Kaether, C., Gerdes, H. H. (1995). Visualization of protein transport along the secretory pathway using green fluorescent protein. FEBS Lett. 369, 267-271
Wang S. X., Hazelrigg, T (1994) Implications for bed mRNA localization from spatial distribution of exu protein in Drosophila oogenesis. Nature (London) 369,400-403

### Example 13

### Aeroporation of Plant Cells

The aeroporation method was used on different preparations of plant cells using DNA vectors coding for β-glucuronidase (GUS), and the pDsRed1-C1 vector which codes for red fluorescent protein. Cells from tobacco leaves cultured from 3-5 days could be transfected with GUS. Aeroporation of maize over the pressure range 5-7 MPa (50-70 Barr) indicated that higher pressures give higher levels of transfection. Aeroporation of tobacco and maize leaves using the red fluorescent protein vector showed apparent transfection levels of 45-55% and 30-35% respectively. Cultures of maize and tobacco cells stably transfected with GFP have also been established.

Previous work using aeroporation to transfect plant cells used TMR-dextran, GFP and β-galactosidase vectors as reporter molecules.

The plant experiments were undertaken using vectors coding for glucuronidase (GUS) that were specifically designed for expression in plants, one designed for dicotyledons and the other for monocotyledons both available from the John Innes Centre (Norwich). GUS assay substrates suitable for both histochemical, spectrophotomentric and fluorimetric analysis are commercially available.

Plants

Tobacco (N. tabacum) and maize (Z. mays) plants were grown in the greenhouse for about 6-7 weeks. The plant tissues used were tobacco and maize leaves (˜1.0 cm long).

Plant Cell Samples

Plant tissues were sterilised (Hall, 1999) and then chopped finely into 1-2 mm cubes. The chopped fragments were either used directly for aeroporation or cultured in a Petri-dish containing 10 ml of MS or B5 culture medium (Hall, 1999) and incubated for 36-48 hours at 24-26° C. on an orbital shaker (140 rpm).

A single cell suspension was prepared from the cultured fragments cultured by using a sterile sieve (mesh 0.5-1.0 mm) to remove all the clumped plant material from the cell suspension. The remaining cell suspension was centrifuged for 5 mins at 750 g. After centrifugation, the pellet was resuspended in the appropriate culture medium followed by incubation at 25° C. The media used was MS culture medium supplemented by 4.5 μM of 2,4-D (Gamborg et al., 1979). Cells were seeded at a density of 2.5×10³ cells/ml in a total volume of 10 ml. Plant cell suspension cultures were maintained in an incubator at 25° C.

Vectors

TMR dextran (70 kdaltons) was used as in indicator that a hole had been created in the cell membrane. The diameter of this molecule is about 5.4 nm.

For dicotyledon transformation with GUS, the pJIT58 vector (5.2 kb) was used (FIG. 4), while for monocotyledon transformation with GUS, the pAL145 vector (6.98 kb) was used (FIG. 6).

PDsRed1-C1 vector expressing red fluorescent protein was used to transfect both monocotyledons and dicotyledons plants (FIG. 10).

Aeroporation Protocolfor Plant Cells

A suspension of cells 4×10⁴ in a volume of 1.0 ml MS medium in a FACS tube was placed in the pressure chamber of the aeroporator and then pressurised to 7 MPa (70 Barr) for 15 minutes and then rapidly de-pressurised. The whole process was carried out at room temperature (20-22° C.). After the aeroporation cycle finished, the cells were taken from the aeroporator and transferred into a microcentrifuge tube. The cells were centrifuged once for 5 mins at 218×g and the pellet was resuspended into 1 ml of culture medium The cell suspension was transferred into a 24-well plate and cultured (25° C.) for 48-72 hours, for expression of DNA.

Microscopic Analysis of Plant Cells

GUS staining (Gallagher S. R., 1992)
Wash 5×10⁴ transfected cells once with phosphate buffered saline (PBS)
Transfer cells to a poly-L-lysine coated slide (slides washed with 70% ethanol+6 ml lysine solution for 1 hour, then rinse 9 times with dd. H₂O). Allow cells to attach for 15 mins and remove the excess liquid
Fix cells with fixative (PBS containing 2% formaldehyde & 0.05% glutaraldehyde), for 5 mins at room temperature
Wash once with PBS
Add stain solution with X-Gluc (1 mg/ml) and incubate overnight (16 hours) at 37° C.
Rinse the cells carefully with PBS and observe under an inverted microscope using the same focus for all samples

Cleavage of the substrate 4-MUG (4-methylumbelliferyl β-D glucuronide) by β-glucuronidase activity leads to the generation of the fluorogenic product 4-MU, which can be visualised with UV light. The protocol used is as described by Gallagher (1989).

Non-Destructive Assay Using MUG in Tissue Culture Media

4-MUG does not appear to be toxic during short incubation periods (up to 2 days), a non-toxic staining procedure in tissue culture media has been developed (Gould and Smith, 1989). Due to the leakage of β-glucuronidase from cultured plant tissues into the mediurn, GUS expression can be analysed in the spent media after transfer of the material to the medium. Alternatively, suspension cultures can be stained directly without destruction of the material.

Assay Protocol

Culture material for 2 days in liquid or agar medium containing 2 mM 4-MUG
Incubate overnight at 30-37° C. The temperature depends on promoter strength
Transfer to new medium
Add 10-30 μl 0.3 M Na₂CO₃ to the tissue
Evaluate staining after 20 mins under UV light
   Results
   Transfection Patterns in Plant Tissue Samples

A suspension of cultured tobacco cells was transfected with GUS pJIT58 vector) using the aeroporator and the transfected cells were visualised using either (A) X-gluc substrate or (B) MUG substrate. The cells were treated for 1 cycle of 15 mins in the aeroporator and the pressure used was 7 MPa (70 Barr).

A suspension of cultured maize cells was transfected with GUS (pAL145 vector) using the aeroporator and visualised using MJG substrate. The cells were treated for 1 cycle of 15 mins in the aeroporator and the pressure used was (A) 5 MPa (50Barr), (B) 6 MPa (60 Barr) and (C) 7 MPa (70 Barr). Untreated controls showed no fluorescence.

From the results, it is clear that from using both fluorescent and non-fluorescent substrates that suspension tobacco cells can be transfected with GUS, using the aeroporation method. The transfection levels obtained were estimated as about 20%. Suspension maize cells were also transfected using the vector designed for expression in monocotyledons. Aeroporation of maize over the pressure range 5-7 MPa (50-70 Barr) indicated that higher pressures give higher levels of transfection.

Transfection experiments using the DsRed1 vector below gave results indicating that aeroporation gave about 50% of cell transfection in the case of suspension tobacco cells, while for cultured cells maize slightly lower transfection levels of about 35% were obtained.

Cultured tobacco leaf cells and cultured maize leaf cells transfected with DsRed1 after 5 days of culture. The pressure used in the aeroporator was 7 MPa (70 Barr) and the cells were treated for 1 cycle of 15 minutes; the gas used was air. Untreated controls showed no red fluorescence.

The preferred pressures are 5-8 MPa (50-80 Barr) using one or more 15 minute cycles in order to maximize transfection and cell yield. Cultures of tobacco and maize leaf cells stably transfected with GFP are capable of growth over at least a 4 week period in non-selective medium.

References

Bevan M. (1984) Nucleic Acid Research 12:8711
Gallagher S. R., (1992) GUS protocols: Using the GUS gene as a Reporter of Gene Expression, 115-120
Gallagher, S. R.,(1989) Spectrophotometric and fluorimetric quantitation of DNA and RNA in solution. Current Protocols in Molecular Biology,A3.9-A3.15
Gamborg O. L., Shyluk J. P., Fowke L. C., Wetter L. R, and Evans D. (1979). Z. Pflanzenphysiol., 95, 255
Gorman C,. (1985). In DNA cloning; A practical Approach, Vol. II, Ed. D. M. Glover, (IRL Press, Oxford, UK), pp. 143-190
Gould, J. H., and Smith, R. H. (1989). A non-destructive assay for GUS in the media of plant tissue cultures. Plant Molecular Biology Rep. 7:209-216
Hall, R. D. (1999). Plant Cell Culture Protocols-Methods in Molecular Biology, 11, 10-17
Jefferson R. A., Kavanagh T. A., and Bevan M. W., (1987), GUS fuisions: β-glucuronidase as a sensitive and versatile gene fusion marker, EMBO J. 6 3901-3908
Lacey A. J. (1989), Fluorescence microscopy, Light microscopy in Biology: A practical approach, Edited by A. J. Lacey
Martin T., Schmidt R., Altmann T., Willmitzer L., Frommer W., Non-destructive assay systems for β-glucuronidase activity in higher plants. Plant Mol. Biol. Rep., in press
Matz, M. V., et al. (1999) Nature Biotechnology 17:969-973
Ploem J. S., (1989), Fluorescence microscopy, Light microscopy in Biology: A practical approach. Edited by A. J. Lacey.

### Example 14

### E. coli Transfection

Growth Conditions of Escherichia coli Cells (E. coli Cells)

E. coli cells were first grown in laurina broth (LB) at 37° C. in a cooled orbital incubator overnight and then streaked onto LB agar plates.

For transformation a single colony was picked from a plate, using a sterile toothpick and 10 ml of LB media was inoculated and grown overnight at 37° C. The next morning 100 μl of cells were removed and added to another 10 ml of media and incubated for 2 hours.

Method of Transfection Using High Pressure Aeroporation

The cells were transformed using the aeroporation procedure as follows:
1× phosphate buffered saline (PBS) was used as the transfection medium and as the washing medium
Cells were counted (approx. 0.5-1×10⁶ cells/ml)
Cells were washed in 1.0 ml of sterile double distilled H₂O by centrifuging at 1300 rpm for 5 mins (2×).
Washed in ice cold 1×PBS
Cells re-suspended in cold 1×PBS and transferred into a FACS tube.
0.5 μl of macromolecules was added into the solution
Tube was placed into the aeroporator chamber and the chamber was closed off.
The air outlet was closed off and the pressure adjusted as required.
The air inlet was opened and pressurisation was allowed to take place for 15 mins.
The chamber was de-pressurised by closing the air inlet and opening the air outlet
The FACS tube was removed from the chamber
The cells were spun at 1300 rpm and then re-suspended in media where the cells were allowed to grow to exponential phase (If Dextrans are being used, the analysis is done immediately after aeroporation).
Cells were prepared for analysis.

Transformation of E. coli by aeroporation was conducted using several commercially available vectors.

TMR dextran was also used for these experiments.

DNA Isolation of Aeroporated Transformed Cells

The Quiagen Endo toxin free Midi Kit was used to isolate the DNA following the manufacturer's instructions.

Results

Transformation of E. coli cells was successful using 1×PBS.

After Transformation cells were grown overnight in selective media and the DNA isolated.

TABLE 9
Transformation of E. coli using different vectors and
1x PBS as the Transfection Media

| | Efficiency of colonies forming on selective | | |
|---|---|---|---|
| | media at different pressures (Barr/0.1 MPa) | | |
| Vectors used | 40 | 50 | 60 |
| pEGFP-C1 | xxxxx | xxxxx | xxxx |
| pEGFP | xxxxx | xxxxx | xx |
| pDsRED2-N1 | xxxx | xxxxx | xxx |
| pCMV·SPORT- | xxxxx | xxxxx | xx |
| βgal | | | |
| pEYFP-C1 | xxx | xx | x |
| pEYFP-N1 | xxx | xxxx | xx |

Key: x very poor growth
xx poor growth
xxx good growth
xxxx good to excellent growth
xxxxx Excellent growth

TMR dextran was also used to investigate transformation using 1×PBS as the transfection media.

TABLE 10
Observations of cells transformed by aeroporation using 1x PBS after
16 hours incubation at 37° C. in a cooled orbital shaking incubator.

| | | | Bacterial growth in LB |
|---|---|---|---|
| | Antibiotic | | selective media after |
| Vector used | plate type | Colonies | transformation |
| pEGFP-C1 | Kanamycin | Yes | Yes |
| pCMV·SPORT- | Ampicillin | Yes | Yes |
| βgal | | | |
| pDsRED2-N1 | Kanamycin | Yes | Yes |
| pEYFP-C1 | Kanamycin | Yes | Yes |
| pEYFP-N1 | Kanamycin | Yes | Yes |
| pEGFP | Ampicillin | Yes | Yes |

From the above results it is clear that E. coli transformation has been successful. All indications show that the aeroporation method is suitable for bacterial transformation leading to DNA isolation.

References

Bell H., Kimber W. L., Li M., Wiftle I. R., Neuroreport, 9 (5), pp.793-798,1998 Fenton M., Bone N., Sinclair A. J., Journal of Immunological Methods, 212(1), pp41-48, 1998
Mascarenhas L., Stripecke R, Case S. S., Xu D. K., Weinberg K. I., Kohn D. B., Blood, 92(10), pp 3537-3545, 1998

### Example 15

### B. subtilis Transfection

Materials

The following materials were employed in this Example:
0.4% Trypan blue in PBS
Sterile distilled water
Sterile 1×PBS
Shuttle vector—JM110 (pHB201)
LB Media
Aeroporator
B. Subtilis (1012M15)
Erythromycin
   Growth of Bacillus subtilis and Aeroporation
Inoculate solid LB media with B. Subtilis using a sterile disposable loop. Incubate at 37° C. overnight
Pick off a single colony and inoculate 10 ml of LB broth and grow overnight in a cooled shaking incubator at 185 rpm
In the morning inoculate a fresh 10 ml of LB with 100 μl of the overnight culture. Incubate at 37° C. for 2 hrs
Cool the B. subtilis on ice for 10 mins and at the same time cool the MCC tubes
Remove 1 ml of liquid culture and add to a cold MCC tube spin at 1000 rpm for five mins
Remove supernatant and then add 1 ml of ice cold sterile water and spin at 1000 rpm (twice)
Remove supernatant and add 1 ml of ice cold 1×PBS spin at 1000 rpm for 5 mins and then remove the supernatant and the add 1 ml of fresh ice cold 1×PBS to the cells and then add 0.5 μl of DNA and then mix and then add to a cold tube
Take tube to the aeroporator and pressurise the chamber for 15 mins
Depressurise and remove tube
Remove cells from the tube and return to the cooled MCC tube
Wash cells as above
Wash in 1×PBS for 5 mins at 1000 rpm and remove the supernatant
Add 1 ml of warm (37° C.) LB media
Perform serial dilutions (optional)
Plate out on selective media, in this case erythromycin
Incubate at 37° C. for 16 hours

Results

TABLE 11
Observation of Bacillus subtilis colonies on selective and non-selective
colonies after aeroporation

| | | | Cells + | Cells no |
|---|---|---|---|---|
| | Cells + | Cells | DNA on | DNA on |
| Pressure | DNA | no DNA | non-selective | non-selective |
| (Barr/0.1 MPa) | on antibiotic | on antibiotic | media | media |
| 40 | Good growth | No | Yes | Yes |
| 50 | Good growth | No | Yes | Yes |
| 60 | Growth | No | Yes | Yes |

The above results in Table 11 demonstrate that B. subtilis was successfully transfected at all pressures and particularly so at 4 and 5 MPa (40 and 50 Barr), since the transfected cells were able to grow on the erythromycin-containing media, in contrast to non-transfected cells.

### Example 16

### Aeroporation of N. tabacum Plant Cells Transfected with FITC-BSA

Aeroporation of N. tabacum (derived leaf mesoplyll tissue) plant cells transfected with FITC-BSA (1 μg/ml). Cells were treated in the aeroporator for 45 min (3 cycles). The first sample was transfected in the presence of air, while the second one was transfected in the presence of oxygen.

Both samples tested positive. In the case of aeroporation in the presence of oxygen alone the expression obtained was higher than that obtained when aeroporation was conducted in the presence of air. Untreated controls showed no fluorescence.

This demonstrates that in some embodiments of the invention, the more soluble the gas employed in the aeroporation method, the more successful the cell transformation.

1. A method for permeabilising a viable cell having a cell wall, comprising:
(a) pressurising a fluid or gel in contact with a surface of the cell; and
(b) depressurising the fluid or gel;
to form at least one hole in a surface of the cell.

2. A method according to claim 1, wherein depressurising the fluid or gel generates bubbles of gas which are capable of forming at least one hole in a surface of the cell.

3. A method according to claim 1, wherein the reduction in pressure in step (b) is 2 MPa (20 Bar) or more.

4. A method according to claim 3, wherein the reduction in pressure in step (b) is from 2-11 MPa (20 Bar to 110 Bar).

5. A method according to claim 4, wherein the reduction in pressure in step (b) is from 5-11 MPa (50 Bar to 110 Bar).

6. A method according to claim 1, wherein the fluid or gel is depressurised in step (b) to substantially atmospheric pressure (about 1 Bar).

7. A method according to claim 1, wherein the hole in the surface of the cell comprises a hole in the cell membrane.

8. A method according to claim 1, wherein the pressure is reduced in step (b) over an interval of less than 10 seconds.

9. A method according to claim 1, wherein the fluid or gel is pressurised in step (a) for a period of 10 mins or more.

10. A method according to claim 9, wherein the fluid or gel is pressurised in step (a) for a period of 10-20 mins.

11. A method according to claim 10, wherein the fluid or gel is pressurised in step (a) for a period of about 15 mins.

12. A method according to claim 1, wherein the fluid or gel comprises an aqueous liquid.

13. A method according to claim 12, wherein the fluid or gel comprises a buffer or a cell culture medium.

14. A method according to claim 1, wherein a gas in contact with the fluid or gel which is subject to the pressurising has a solubility in the fluid or gel of 1.0×10⁻⁴ mol/l atm or more.

15. A method according to claim 14, wherein the gas has a solubility of 6.0×10⁻⁴ mol/l atm or more.

16. A method according to claim 14, wherein the gas comprises a gas selected from air, oxygen, nitrogen, carbon dioxide, methane, helium, neon, and argon.

17. A method according to claim 1, which method consists of a single pressurising and depressurising cycle, or multiple pressuring and depressurising cycles.

18. A method according to claim 1, wherein the cell is a plant cell, a-fungal cell or a bacterial cell.

19. A method according to claim 18, wherein the cell is a cell from a crop plant.

20. A method according to claim 19, wherein the crop plant is selected from a cereal or pulse, maize, wheat, potato, tapioca, rice, sorghum, millet, cassava, barley, pea, and another root, tuber, or seed crop.

21. A method according to claim 20, wherein the seed crop is selected from oil-seed rape, sugar beet, maize, sunflower, soybean, and sorghum.

22. A method according to claim 18, wherein the plant cell is a cell from a horticultural plant.

23. A method according to claim 22, wherein the horticultural plant is selected from lettuce, endive, vegetable brassicas including cabbage broccoli and cauliflower, carnation, geranium, tobacco, cucurbits, carrot, strawberry, sunflower, tomato, pepper, chrysanthemum, poplar, eucalyptus, and pine.

24. A method according to claim 18, wherein the cell is from a seed producing plant selected from oil-seed plants, cereal seed producing plants and leguminous plants.

25. A method according to claim 24, wherein the oil seed plant is selected from cotton, soybean, safflower, sunflower, oil-seed rape, maize, alfalfa, palm, and coconut.

26. A method according to claim 24 wherein the cereal seed producing plant is selected from corn, wheat, barley, rice, sorghum, and rye, and other grain seed producing plants.

27. A method according to claim 24, wherein the leguminous plant is selected from peas and beans, including guar, locust bean, fenugreek, soybean, garden beans, cowpea, mungbean, lima bean, fava bean, lentils, and chickpea.

28. A method according to claim 18, wherein the cell is a cell from a plant selected from corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea Americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats, barley, vegetables, ornamentals, and conifers.

29. A method according to claim 18 wherein the bacterial cell is a gram-positive or gram-negative bacterium.

30. A method according to claim 29, wherein the cell is a cell selected from E. coil, B. subtilis, S. cerevisiae, F. graminearum, S. pombe, Z. mays, and N. tabacum.

31. A method according to claim 1, wherein the cell forms part of a cluster of cells.

32. A method according to claim 31, wherein the cluster is an embryogenic cluster.

33. A method according to claim 1, wherein the cell is a microspore.

34. A method according to claim 33, wherein the cell is a pollen microspore.

35. A method according to claim 1, wherein the temperature of the fluid or gel is up to 37° C.

36. A method according to claim 35, wherein the temperature is from 15-30° C.

37. A method for introducing a substance into a cell having a cell wall, comprising a method according to claim 1, and wherein the at least one hole facilitates entry of the substance into the cell.

38. A method according to claim 37, wherein the fluid or gel comprises the substance.

39. A method according to claim 37, wherein the substance is selected from a biological molecule or a macromolecule.

40. A method according to claim 39, wherein the substance is selected from a nucleic acid including DNA, cDNA, RNA or mRNA

41. A method according to claim 40, wherein the nucleic acid comprises a gene, a plasmid, a chromosome, an oligonucleotide, a nucleotide sequence, a ribozyme or a fragment thereof, or an expression vector.

42. A method according to claim 39, wherein the substance comprises a bio-active molecule, including a protein, a polypeptide, a peptide, an amino acid, a hormone, a polysaccharide, a dye, and a pharmaceutical agent such as drug.

43. A method according to claim 37, wherein the substance has a molecular weight of 100 Daltons or more.

44. A permeabilised cell having a cell wall obtainable by a method as defined in claim 1, wherein the surface of the cell comprises at least one hole which is capable of facilitating the entry of a substance into the cell.

45. A permeabilised cell according to claim 44, wherein the hole comprises a hole in the cell membrane.

46. A permeabilised cell according to claim 44, wherein the cell wall of the cell is substantially intact.

47. Use of a depressurisation means to permeabilise a cell and/or to introduce a substance into a cell, wherein the cell has a cell wall, and the depressurisation means is used to reduce the pressure applied to a fluid or gel comprising the cell by a step of 2 MPa (20 Bar) or more.

48. An apparatus for introducing a substance into a cell having a cell wall, using a method as defined in claim 1, which apparatus comprises:
(a) an inlet for introducing a gas;
(b) a pressure chamber into which the inlet feeds, which chamber is of substantially geometrical cross section;
(c) a compartment within the pressure chamber for containing the cell in a fluid or gel;
(d) optionally a pressure gauge for monitoring the pressure in the pressure chamber; and
(e) an outlet for releasing gas from the pressure chamber;
wherein both the inlet and the outlet comprise a valve for isolating the pressure chamber during pressurisation.

49. An apparatus according to claim 48, wherein the inlet and outlet comprise inlet and outlet tubes.

50. An apparatus according to claim 49 wherein the diameter of the inlet tube and/or the outlet tube is from 2-4 mm.

51. An apparatus according to claim 48, wherein the geometrical cross section of the pressurisation chamber is substantially cylindrical.

52. An apparatus according to claim 48, wherein the compartment for containing the cell in a fluid or gel comprises substantially the entire internal surface of the pressure chamber.

53. An apparatus according to claim 52, wherein the internal surface of the pressure chamber comprises a physiologically acceptable coating.

54. An apparatus according to claim 48, wherein the compartment for containing the cell in a fluid or gel comprises a receptacle positioned adjacent to an internal surface of the pressure chamber.

55. An apparatus according to claim 54, wherein the receptacle is supported by the internal surface of the pressure chamber.

56. An apparatus according to claim 54, wherein the internal surface of the receptacle comprises a physiologically acceptable coating.

57. An apparatus according to claim 48, wherein the valve in the inlet and/or the outlet comprises a needle valve.
